(19) United States
(12) Patent Application Publication
(10) Pub. No.: US 2012/0264632 A1
(43) Pub. Date: Oct. 18, 2012

(54) Methods for Determining Sequence Variants Using Ultra-Deep Sequencing

(76) Inventors: John Harris Leamon, Guilford, CT (US); William Lun Lee, Madison, CT (US); Jan Fredrik Simons, new Haven, CT (US); Brian Desany, East Haven, CT (US); Michael Todd Ronan, New Haven, CT (US); James Drake, Branford, CT (US); Kenton Lohman, Guilford, CT (US); Michael Egholm, Madison, CT (US); Jonathan Rothberg, Guilford, CT (US)

(21) Appl. No.: 13/443,629

(22) Filed: Apr. 10, 2012

Related U.S. Application Data
(63) Continuation of application No. 11/104,781, filed on Apr. 12, 2005, now abandoned.

(51) Int. Cl. C40B 30/04; C12Q 1/68

(52) U.S. Cl. 506/9; 435/6.11

(57) ABSTRACT

The claimed invention provides for new sample preparation methods enabling direct sequencing of PCR products using pyrophosphate sequencing techniques. The PCR products may be specific regions of a genome. The techniques provided in this disclosure allows for SNP (single nucleotide polymorphism) detection, classification, and assessment of individual allelic polymorphisms in one individual or a population of individuals. The results may be used for diagnostic and treatment of patients as well as assessment of viral and bacterial population identification.

### RELATED APPLICATIONS

This application is a continuation of U.S. Ser. No. 11/104,781, filed Apr. 12, 2005, which is herein incorporated by reference in its entirety.

### FIELD OF THE INVENTION

The invention provides methods, reagents and systems for detecting and analyzing sequence variants including single nucleotide polymorphisms (SNPs), insertion/deletion variant (referred to as “indels”) and allelic frequencies, in a population of target polynucleotides in parallel. The invention also relates to a method of investigating by parallel pyrophosphate sequencing nucleic acids replicated by polymerase chain reaction (PCR), for the identification of mutations and polymorphisms of both known and unknown sequences. The invention involves using nucleic acid primers to amplify a region or regions of nucleic acid in a target nucleic acid population which is suspected of containing a sequence variant to generate amplicons. Individual amplicons are sequenced in an efficient and cost effective manner to generate a distribution of the sequence variants found in the amplified nucleic acid.

### BACKGROUND OF THE INVENTION

Genomic DNA varies significantly from individual to individual, except in identical siblings. Many human diseases arise from genomic variations. The genetic diversity amongst humans and other life forms explains the heritable variations observed in disease susceptibility. Diseases arising from such genetic variations include Huntington's disease, cystic fibrosis, Duchenne muscular dystrophy, and certain forms of breast cancer. Each of these diseases is associated with a single gene mutation. Diseases such as multiple sclerosis, diabetes, Parkinson's, Alzheimer's disease, and hypertension are much more complex. These diseases may be due to polygenic (multiple gene influences) or multifactorial (multiple gene and environmental influences) causes. Many of the variations in the genome do not result in a disease trait. However, as described above, a single mutation can result in a disease trait. The ability to scan the human genome to identify the location of genes which underlie or are associated with the pathology of such diseases is an enormously powerful tool in medicine and human biology.

Several types of sequence variations, including insertions and deletions (indels), differences in the number of repeated sequences, and single base pair differences (SNPs) result in genomic diversity. Single base pair differences, referred to as single nucleotide polymorphisms (SNPs) are the most frequent type of variation in the human genome (occurring at approximately 1 in 10.sup.3 bases). A SNP is a genomic position at which at least two or more alternative nucleotide alleles occur at a relatively high frequency (greater than 1%) in a population. A SNP may also be a single base (or a few bases) insertion/deletion variant (referred to as “indels”). SNPs are well-suited for studying sequence variation because they are relatively stable (i.e., exhibit low mutation rates) and because single nucleotide variations (including insertions and deletions) can be responsible for inherited traits. It is understood that in the discussion above, the term SNP is also meant to be applicable to “indel” (defined below).

Polymorphisms identified using microsatellite-based analysis, for example, have been used for a variety of purposes. Use of genetic linkage strategies to identify the locations of single Mendelian factors has been successful in many cases (Benomar et al. (1995), Nat. Genet., 10:84-8; Blanton et al. (1991), Genomics, 11:857-69). Identification of chromosomal locations of tumor suppressor genes has generally been accomplished by studying loss of heterozygosity in human tumors (Cavenee et al. (1983), Nature, 305:779-784; Collins et al. (1996), Proc. Natl. Acad. Sci. USA, 93:14771-14775; Koufos et al. (1984), Nature, 309:170-172; and Legius et al. (1993), Nat. Genet., 3:122-126). Additionally, use of genetic markers to infer the chromosomal locations of genes contributing to complex traits, such as type I diabetes (Davis et al. (1994), Nature, 371:130-136; Todd et al. (1995), Proc. Natl. Acad. Sci. USA, 92:8560-8565), has become a focus of research in human genetics.

Although substantial progress has been made in identifying the genetic basis of many human diseases, current methodologies used to develop this information are limited by prohibitive costs and the extensive amount of work required to obtain genotype information from large sample populations. These limitations make identification of complex gene mutations contributing to disorders such as diabetes extremely difficult. Techniques for scanning the human genome to identify the locations of genes involved in disease processes began in the early 1980s with the use of restriction fragment length polymorphism (RFLP) analysis (Botstein et al. (1980), Am. J. Hum. Genet., 32:314-31; Nakamura et al. (1987), Science, 235:1616-22). RFLP analysis involves southern blotting and other techniques. Southern blotting is both expensive and time-consuming when performed on large numbers of samples, such as those required to identify a complex genotype associated with a particular phenotype. Some of these problems were avoided with the development of polymerase chain reaction (PCR) based microsatellite marker analysis. Microsatellite markers are simple sequence length polymorphisms (SSLPs) consisting of di-, tri-, and tetra-nucleotide repeats.

Other types of genomic analysis are based on use of markers which hybridize with hypervariable regions of DNA having multiallelic variation and high heterozygosity. The variable regions which are useful for fingerprinting genomic DNA are tandem repeats of a short sequence referred to as a mini satellite. Polymorphism is due to allelic differences in the number of repeats, which can arise as a result of mitotic or meiotic unequal exchanges or by DNA slippage during replication.

Each of these current methods have significant drawbacks because they are time consuming and limited in resolution. While DNA sequencing provides the highest resolution, it is also the most expensive method for determining SNPs. At this time, the determination of SNP frequency among a population of 1000 different samples is very expensive and the determination of SNP frequency among a population of 100,000 samples is prohibitive.

### BRIEF SUMMARY OF THE INVENTION

The invention relates to methods of diagnosing a number of sequence variants (e.g., allelic variants, single nucleotide polymorphism variants, indel variants) by the identification of specific DNA. Current technology allows detection of SNPs, for example, by polymerase chain reaction (PCR). However, SNPs detection by PCR requires the design of special PCR primers which hybridize to one type of SNP and not another type of SNP. Furthermore, although PCR is a powerful technique, the specific PCR of alleles require prior knowledge of the nature (sequence) of the SNP, as well as multiple PCR runs and analysis on gel electrophoresis to determine an allelic frequency. For example, an allelic frequency of 5% (i.e., 1 in 20) would require at a minimum 20 PCR reactions for its detection. The amount of PCR and gel electrophoresis needed to detect an allelic frequency goes up dramatically as the allelic frequency is reduced, for example to 4%, 3%, 2% or 1% or less.

None of the current methods has provided a simple and rapid method of detecting SNP, including SNP of low abundance, by identification of specific DNA sequence.

We have found that a two stage PCR technique coupled with a novel pyrophosphate sequencing technique would allow the detection of sequence variants (SNP, indels and other DNA polymorphisms) in a rapid, reliable, and cost effective manner. Furthermore, the method of the invention can detect sequence variants which are present in a DNA sample in nonstoichmetric allele amounts, such as, for example, DNA variants present in less than 50%, less than 25%, less than 10%, less than 5% or less than 1%. The techniques may conveniently be termed “ultradeep sequencing.”

According to the present invention there is provided a method for diagnosing a sequence variant [symbol] such as an allelic frequency, SNP frequency, indel frequency) by specific amplification and sequencing of multiple alleles in a nucleic acid sample. The nucleic acid is first subjected to amplification by a pair of PCR primers designed to amplify a region surrounding the region of interest. Each of the products of the PCR reaction (amplicons) is subsequently further amplified individually in separate reaction vessels using EBCA (Emulsion Based Clonal Amplification). EBCA amplicons (referred to herein as second amplicons) are sequenced and the collection of sequences, from different emulsion PCR amplicons, is used to determine an allelic frequency.

One embodiment of the invention is directed to a method for detecting a sequence variant in a nucleic acid population. The sequence variant may be a SNP, an indel, a sequence nucleotide frequency, or an allelic frequency or a combination of these parameters. The method involves the steps of amplifying a DNA segment common to the nucleic acid population with a pair of nucleic acid primers that define a locus to produce a first population of amplicons each comprising the DNA segment. Each member of the first population of amplicons is clonally amplified to produce a population of second amplicons where each population of second amplicons derives from one member of the first population of amplicons. The second amplicons are immobilized to a plurality of mobile solid supports such that each mobile solid support is attached to one population of the second amplicons. The nucleic acid on each mobile solid support is sequenced to produce a population of nucleic acid sequences—one sequence per mobile solid support. A sequence variant, an allelic frequency, a SNP or an indel may be determined from the population of nucleic acid sequences.

Another embodiment of the invention is directed to a method of identifying a population with a plurality of different species of organisms. The method involves isolating a nucleic acid sample from the population so that the nucleic acid sample is a mixture of DNA from each member of the population. Then, a nucleotide frequency of a nucleic acid segment of a locus common to all organisms in the population may be generated from the method of the previous paragraph. The locus is required to have a different sequence (allele) for each different species. That is, each species should have at a different nucleic acid sequence at the locus. The allelic frequency may be determind from the incidence of each type of nucleotide at the locus. A distribution of organisms in the population may be determined from the allelic frequency.

In a preferred embodiment, the method of the invention is used to determine SNPs and indels distribution in a nucleic acid sample. The target population of nucleic acid may be from an individual, a tissue sample, a culture sample, a environmental sample such as a soil sample (See, e.g., Example 5 and Example 3), or any other types of nucleic acid sample which contains at least two different nucleic acids with each nucleic acid representing a different allele.

The method of the invention may be used to analyze a tissue sample to determine its allelic composition. For example, tumor tissues may be analyzed to determine if they contain the same allele at the locus of an oncogene. Using this method, the percentage of cells in the tumor with an activated or mutated oncogene and the total amount of tumor DNA in a DNA sample may be determined.

### BRIEF DESCRIPTION OF THE FIGURES

FIG. 1 depicts a schematic of one embodiment of a bead emulsion amplification process.

FIG. 2 depicts schematic of ultradeep sequencing method.

FIG. 3 depicts quality assessment of amplicons produced with primer pairs SAD1F/R-DD14 (panel A), SAD1F/R-DE15 (panel B) and SAD1F/R-F5 (panel C). Analysis was performed on a BioAnalyzer DNA 1000 BioChip with the center peaks representing the PCR products and the flanking peaks reference size markers. Each peak was measured to be within 5 bp of the theoretical size which ranged from 156-181 base pairs.

FIG. 4 depicts nucleotide frequencies (frequency of non-matches) in amplicons representing two distinct alleles in the MHC II locus were mixed in approximate ratios (C allele to T allele) of 1:500 (A) and 1:1000 (B), or T allele only (A), clonally amplified and sequenced on 454 Life Sciences' sequencing platform. Each bar represents the frequency of deviation from the consensus sequence and are color-coded according to the resulting base substitution (red=A; green=C; blue=G; yellow=T).

FIG. 5 depicts the same data as presented in FIGS. 4B and 4C, however after background subtraction using the T allele only sample presented in FIG. 4A.

FIG. 6 depicts various ratios of C to T alleles from the DD14 HLA locus were mixed and sequenced on the 454 platform to determine dynamic range. The experimentally observed ratios are plotted against the intended ratios (abscissa). The actual number of sequencing reads for each data point is summarized in Table 1

FIG. 7 A: A graphical display showing the location of the reads mapping to the 1.6 Kb 16S gene fragment indicating roughly 12,000 reads mapping to the first 100 bases of the 16S gene. B: shows similar results as 7A except with the V3 primers which maps to a region around base 1000. C: shows locations of the reads where both V1 and V3 primers are used.

FIG. 8 depicts a phylogentic tree which clearly discriminates between the V1 (shorter length on left half of figure) and the V3 (longer length on right half of figure) sequences in all but 1 of the 200 sequences.

### DETAILED DESCRIPTION OF THE INVENTION

The invention relates to methods of detecting one or more sequence variants by the identification of specific DNA. Sequence variants encompass any sequence differences between two nucleic acid molecules. As such, sequence variants is understood to also refer to, at least, single nucleotide polymorphisms, insertion/deletions (indels), allelic frequencies and nucleotide frequencies—that is, these terms are interchangeable. While different detection techniques are discussed throughtout this specification using specific examples, it is understood that the process of the invention is equally applicable to the detection of any sequence variants. For example, a discussion of a process for detecting SNPs in this disclosure is also applicable to a process for detecting indels or nucleotide frequencies.

This process of the invention may be used to amplify and sequence specific targeted templates such as those found within genomes, tissue samples, heterogeneous cell populations or environmental samples. These can include, for example, PCR products, candidate genes, mutational hot spots, evolutionary or medically important variable regions. It could also be used for applications such as whole genome amplification with subsequent whole genome sequencing by using variable or degenerate amplification primers.

To date, sequencing targeted templates have required preparation and sequencing entire genomes of interest or prior PCR amplification of a region of interest and the sequencing of that region. The methods of the invention allow SNP sequencing to be performed at substantially greater depth than currently provided by existing technology.

In this disclosure, single nucleotide polymorphism (SNP) may be defined as a SNP that exists in at least two variants where the least common variant is present in at least 1% of the population (Wang et al., 1998 Science 280:1077-1082). It is understood that the methods of the disclosure may be applied to “indels.” Therefore, while the instant disclosure makes references to SNP, it is understood that this disclosure is equally applicable if the term “SNP” is substituted with the term “indel” at any location.

As used herein, the term “indel” is intended to mean the presence of an insertion or a deletion of one or more nucleotides within a nucleic acid sequence compared to a related nucleic acid sequence. An insertion or a deletion therefore includes the presence or absence of a unique nucleotide or nucleotides in one nucleic acid sequence compared to an otherwise identical nucleic acid sequence at adjacent nucleotide positions. Insertions and deletions can include, for example, a single nucleotide, a few nucleotides or many nucleotides, including 5, 10, 20, 50, 100 or more nucleotides at any particular position compared to the related reference sequence. It is understood that the term also includes more than one insertion or deletion within a nucleic acid sequence compared to a related sequence.

Poisson statistics indicates that the lower limit of detection (i.e., less than one event) for a fully loaded 60 mm×60 mm picotiter plate (2×10⁶ high quality bases, comprised of 200,000×100 base reads) is three events with a 95% confidence of detection and five events with a 99% confidence of detection (see Table 1). This scales directly with the number of reads, so the same limits of detection hold for three or five events in 10,000 reads, 1000, reads or 100 reads. Since the actual amount of DNA read is higher than the 200,000, the actual lower limit of detection is expected to at an even lower point due to the increased sensitivity of the assay. For comparison, SNP detection via pyrophosphate based sequencing has reported detection of separate allelic states on a tetraploid genome, so long as the ratio least frequent allele is present in 10% or more of the population (Rickert et al., 2002 BioTechniques. 32:592-603). Conventional fluorescent DNA sequencing is even less sensitive, experiencing trouble resolving 50/50 (i.e., 50%) heterozygote alleles (Ahmadian et al., 2000 Anal. BioChem. 280:103-110).

TABLE 1
Probability of detecting zero or one or more events,
based on number of events in total population.

| | | Percent chance |
|---|---|---|
| | Percent chance | of detecting |
| Copies of | of detecting | one or |
| Sequence | zero copies | more copies |
| 1 | 36.8 | 63.2 |
| 2 | 13.5 | 86.5 |
| 3 | 5.0* | 95.0* |
| 4 | 1.8 | 98.2 |
| 5 | 0.7** | 99.3** |
| 6 | 0.2 | 99.8 |
| 7 | 0.1 | 99.9 |
| 8 | 0.0 | 100.0 |
| 9 | 0.0 | 100.0 |
| 10 | 0.0 | 100.0 |

“*” indicates that probability of failing to detect three events is 5.0%., thus the probability of detecting said event is 95%; similarly,
“**” reveals that that probability of detecting one or more events that occur 5 times is 99.3%.

As a result, utilizing an entire 60×60 mm picotiter plate to detect a single SNP permits detection of a SNP present in only 0.002% of the population with a 95% confidence or in 0.003% of the population with 99% confidence. Naturally, multiplex analysis is of greater applicability than this depth of detection and Table 2 displays the number of SNPs that can be screened simultaneously on a single picotiter plate, with the minimum allelic frequencies detectable at 95% and 99% confidence.

TABLE 2

| | | Frequency | Frequency |
|---|---|---|---|
| | | of SNP in | of SNP in |
| SNP | Number | population with | population with |
| Classes | of Reads | 95% confidence | 99% confidence |
| 1 | 200000 | 0.002% | 0.003% |
| 2 | 10000 | 0.030% | 0.050% |
| 5 | 4000 | 0.075% | 0.125% |
| 10 | 2000 | 0.15% | 0.25% |
| 50 | 400 | 0.75% | 1.25% |
| 100 | 200 | 1.50% | 2.5% |
| 150 | 133 | 2.25% | 3.75% |
| 200 | 100 | 3.0% | 5.0% |
| 500 | 40 | 7.5% | 12.5% |
| 1000 | 20 | 15.0% | 25.0% |

One advantage of the invention is that a number of steps, usually associated with sample preparation (e.g., extracting and isolating DNA from tissue for sequencing) may be eliminated or simplified. For example, because of the sensitivity of the method, it is no longer necessary to extract DNA from tissue using traditional technique of grinding tissue and chemical purification. Instead, a small tissue sample of less than one microliter in volume may be boiled and used for the first PCR amplication. The product of this solution amplification is added directly to the emPCR reaction. The methods of the invention therefore reduce the time and effort and product loss (including loss due to human error).

Another advantage of the methods of the invention is that the method is highly amenable to multiplexing. As discussed below, the bipartite primers of the invention allows combining primer sets for multiple genes with identical pyrophosphate sequencing primer sets in a single solution amplification. Alternatively, the product of multiple preparations may be placed in a single emulsion PCR reaction. As a result, the methods of the invention exhibit considerable potential for high throughput applications.

One embodiment of the invention is directed to a method for determining an allelic frequency (including SNP and indel frequency). In the first step, a first population of amplicons is produced by PCR using a first set of primers to amplify a target population of nucleic acids comprising the locus to be analyzed. The locus may comprise a plurality of alleles such as, for example, 2, 4, 10, 15 or 20 or more alleles. The first amplicons may be of any size, such as, for example, between 50 and 100 bp, between 100 bp and 200 bp, or between 200 bp to 1 kb. One advantage of the method is that knowledge of the nucleic acid sequence between the two primers is not required.

In the next step, the population of first amplicons is delivered into aqueous microreactors in a water-in-oil emulsion such that a plurality of aqueous microreactors comprises (1) sufficient DNA to initiate an amplification reaction dominated by a single template or amplicon (2) a single bead, and (3) amplification reaction solution containing reagents necessary to perform nucleic acid amplification (See discussion regarding EBCA (Emulsion Based Clonal Amplification) below). We have found that an amplification reaction dominated by a single template or amplicon may be achieved even if two or more templates are present in the microreactor. Therefore, aqueous microreactors comprising more than one template are also envisioned by the invention. In a preferred embodiment, each aqueous microreactor has a single copy of DNA template for amplification.

After the delivery step, the first population of amplicons is amplified in the microreactors to form second amplicons. Amplification may be performed, for example, using EBCA (which involves PCR) in a thermocycler to produce second amplicons. After EBCA, the second amplicons is bound to the beads in the microreactors. The beads, with bound second amplicons are delivered to an array of reaction chambers (e.g., an array of at least 10,000 reaction chambers) on a planar surface. The delivery is adjusted such that a plurality of the reaction chambers comprise no more than a single bead. This may be accomplished, for example, by using an array where the reaction chambers are sufficiently small to accommodate only a single bead.

A sequencing reaction is performed simultaneously on the plurality of reaction chambers to determine a plurality of nucleic acid sequences corresponding to said plurality of alleles. Methods of parallel sequencing in parallel using reaction chambers are disclosed in another section above and in the Examples. Following sequencing, the allelic frequency, for at least two alleles, may be determined by analyzing the sequences from the target population of nucleic acids. As an example, if 10000 sequences are determined and 9900 sequences read “aaa” while 100 sequences read “aag,” the “aaa” allele may be said to have a frequency of 90% while the “aag” allele would have a frequency of 10%. This is described in more detail in the description below and in the Examples.

One advantage of the invention's methods is that it allows a higher level of sensitivity than previously achieved. If a picotiter plate is used, the methods of the invention can sequence over 100,000 or over 300,000 different copies of an allele per picotiter plate. The sensitivity of detection should allow detection of low abundance alleles which may represent 1% or less of the allelic variants. Another advantage of the invention's methods is that the sequencing reaction also provides the sequence of the analyzed region. That is, it is not necessary to have prior knowledge of the sequence of the locus being analyzed.

In a preferred embodiment, the methods of the invention may detect an allelic frequency which is less than 10%, less than 5%, or less than 2%. In a more preferred embodiment, the method may detect allelic frequencies of less than 1%, such as less than 0.5% or less than 0.2%. Typical ranges of detection sensitive may be between 0.1% and 100%, between 0.1% and 50%, between 0.1% and 10% such as between 0.2% and 5%.

The target population of nucleic acids may be from a number of sources. For example, the source may be a tissue or body fluid from an organism. The organism may be any organism including mammals. The mammals may be a human or a commercially valuable livestock such as cows, sheep, pigs, goats, rabbits, and the like. The method of the invention would allow analysis tissue and fluid samples of plants. While all plants may be analyzed by the methods of the invention, preferred plants for the methods of the invention include commercially valuable crops species including monocots and dicots. In one preferred embodiment, the target population of nucleic acids may be derived from a grain or food product to determine the original and distribution of genotypes, alleles, or species that make up the grain or food product. Such crops include, for example, maize, sweet corn, squash, melon, cucumber, sugarbeet, sunflower, rice, cotton, canola, sweet potato, bean, cowpea, tobacco, soybean, alfalfa, wheat, or the like.

Nucleic acid samples may be collected from multiple organisms. For example, allelic frequency of a population of 1000 individuals may be performed in one experiment analyzing a mixed DNA sample from 1000 individuals. Naturally, for a mixed DNA sample to be representative of the allelic frequency of a population, each member of the population (each individual) must contribute the same (or approximately the same) amount of nucleic acid (same number of copies of an allele) to the pooled sample. For example, in an analysis of genomic allelic frequency, each individual may contribute the DNA from approximately 1.0×10⁶ cells to a pooled DNA sample.

In another embodiment of the invention, the polymorphism in a single individual may be determined. That is the target nucleic acid may be isolated from a single individual. For example, pooled nucleic acids from multiple tissue sample of an individual may be examined for polymorphisms and nucleotide frequencies. This may be useful, for example, for determining polymorphism in a tumor, or a tissue suspected to contain a tumor, of an individual. The method of the invention may be used, for example, to determine the frequency of an activated oncogene in a tissue sample (or pooled DNA from multiple tissue sample) of an individual. In this example, an allelic frequency of 50% or more of activated oncogenes may indicate that the tumor is monoclonal. The presence of less than 50% of an activated oncogene may indicate that the tumor is polyclonal, or that the tissue sample contains a combination of tumor tissue and normal (non-tumor) tissue. Furthermore, in a biopsy of a suspect tissue, the presence of, for example, 1% of an activated oncogene may indicate the presence of an emerging tumor, or the presence of a malignant tumor infiltration.

The target population of nucleic acids may be any nucleic acid including, DNA, RNA and various forms of such DNA and RNAs such as plasmids, cosmids, DNA viral genomes, RNA viral genome, bacterial genomes, mitochondrial DNA, mammalian genomes, plant genomes. The nucleic acid may be isolated from a tissue sample or from an in vitro culture. Genomic DNA can be isolated from a tissue sample, a whole organism, or a sample of cells. If desired, the target population of nucleic acid may be normalized such that it contains an equal amount of alleles from each individual that contributed to the population.

One advantage of the invention is that the genomic DNA may be used directly without further processing. However, in a preferred embodiment, the genomic DNA may be substantially free of proteins that interfere with PCR or hybridization processes, and are also substantially free of proteins that damage DNA, such as nucleases. Preferably, the isolated genomes are also free of non-protein inhibitors of polymerase function (e.g. heavy metals) and non-protein inhibitors of hybridization which would interfere with a PCR. Proteins may be removed from the isolated genomes by many methods known in the art. For instance, proteins may be removed using a protease, such as proteinase K or pronase, by using a strong detergent such as sodium dodecyl sulfate (SDS) or sodium lauryl sarcosinate (SLS) to lyse the cells from which the isolated genomes are obtained, or both. Lysed cells may be extracted with phenol and chloroform to produce an aqueous phase containing nucleic acid, including the isolated genomes, which can be precipitated with ethanol.

The target population of nucleic acid may be derived from sources with unknown origins of DNA such as soil samples, food samples and the like. For example, the sequencing of an allele found in a pathogen in a nucleic acid sample from a food sample would allow the determination the presence of pathogen contamination in the food. Furthermore, the methods of the invention would allow determination of the distribution of pathogenic allele in the food. For example, the methods of the invention can determine the strain (species) or distribution of strains (species) of a particular organism (e.g., bacteria, virus, pathogens) in an environmental sample such as a soil sample (See, Example 5) or a seawater sample.

One advantage of the method is that no a priori knowledge of mutations required for the method. Because the method is based on nucleic acid sequencing, all mutations in one location would be detected. Furthermore, no cloning is required for the sequencing. A DNA sample is amplified in sequenced in a series of step without the need for cloning, subcloning, and culturing of the cloned DNA.

The methods of the invention may be used, for example, for detection and quantification of all variants in viral samples. These viral samples may include, for example, an HIV viral isolate. Other applications of the method include population studies of sequence variants. DNA samples may be collected from a population of organisms and combined and analyzed in one experiment to determine allelic frequencies. The populations of organisms may include, for example, a population of humans, a population of livestock, a population of grain from a harvest and the like. Other uses include detection and quantification of somatic mutations in tumor biopsies (e.g. lung and colorectal cancer) from biopsy comprising a mixed population of tumor and normal cells. The methods of the invention may also be used for high confidence re-sequencing of clinically relevant susceptibility genes (e.g. breast, ovarian, colorectal and pancreatic cancer, melanoma).

Another use for the invention involves identification of polymorphisms associated with a plurality of distinct genomes. The distinct genomes may be isolated from populations which are related by some phenotypic characteristic, familial origin, physical proximity, race, class, etc. In other cases, the genomes are selected at random from populations such that they have no relation to one another other than being selected from the same population. In one preferred embodiment, the method is performed to determine the genotype (e.g. SNP content) of subjects having a specific phenotypic characteristic, such as a genetic disease or other trait.

The methods of the invention may also be used to characterize the genetic makeup of a tumor by testing for loss of heterozygosity or to determine the allelic frequency of a particular SNP. Additionally, the methods may be used to generate a genomic classification code for a genome by identifying the presence or absence of each of a panel of SNPs in the genome and to determine the allelic frequency of the SNPs. Each of these uses is discussed in more detail herein.

A preferred use of the invention is in a high throughput method of genotyping. “Genotyping” is the process of identifying the presence or absence of specific genomic sequences within genomic DNA. Distinct genomes may be isolated from individuals of populations which are related by some phenotypic characteristic, by familial origin, by physical proximity, by race, by class, etc. in order to identify polymorphisms (e.g. ones associated with a plurality of distinct genomes) which are correlated with the phenotype family, location, race, class, etc. Alternatively, distinct genomes may be isolated at random from populations such that they have no relation to one another other than their origin in the population. Identification of polymorphisms in such genomes indicates the presence or absence of the polymorphisms in the population as a whole, but not necessarily correlated with a particular phenotype. Since a genome may span a long region of DNA and may involve multiple chromosomes, a method of the invention for detecting a genotype would need to analyze a plurality of sequence variants at multiple locations to detect a genotype at a reliability of 99.99%.

Although genotyping is often used to identify a polymorphism associated with a particular phenotypic trait, this correlation is not necessary. Genotyping only requires that a polymorphism, which may or may not reside in a coding region, is present. When genotyping is used to identify a phenotypic characteristic, it is presumed that the polymorphism affects the phenotypic trait being characterized. A phenotype may be desirable, detrimental, or, in some cases, neutral. Polymorphisms identified according to the methods of the invention can contribute to a phenotype. Some polymorphisms occur within a protein coding sequence and thus can affect the protein structure, thereby causing or contributing to an observed phenotype. Other polymorphisms occur outside of the protein coding sequence but affect the expression of the gene. Still other polymorphisms merely occur near genes of interest and are useful as markers of that gene. A single polymorphism can cause or contribute to more than one phenotypic characteristic and, likewise, a single phenotypic characteristic may be due to more than one polymorphism. In general multiple polymorphisms occurring within a gene correlate with the same phenotype. Additionally, whether an individual is heterozygous or homozygous for a particular polymorphism can affect the presence or absence of a particular phenotypic trait.

Phenotypic correlation is performed by identifying an experimental population of subjects exhibiting a phenotypic characteristic and a control population which do not exhibit that phenotypic characteristic. Polymorphisms which occur within the experimental population of subjects sharing a phenotypic characteristic and which do not occur in the control population are said to be polymorphisms which are correlated with a phenotypic trait. Once a polymorphism has been identified as being correlated with a phenotypic trait, genomes of subjects which have potential to develop a phenotypic trait or characteristic can be screened to determine occurrence or non-occurrence of the polymorphism in the subjects' genomes in order to establish whether those subjects are likely to eventually develop the phenotypic characteristic. These types of analyses are may be performed on subjects at risk of developing a particular disorder such as Huntington's disease or breast cancer.

One embodiment of the invention is directed to a method for associating a phenotypic trait with an SNP. A phenotypic trait encompasses any type of genetic disease, condition, or characteristic, the presence or absence of which can be positively determined in a subject. Phenotypic traits that are genetic diseases or conditions include multifactorial diseases of which a component may be genetic (e.g. owing to occurrence in the subject of a SNP), and predisposition to such diseases. These diseases include such as, but not limited to, asthma, cancer, autoimmune diseases, inflammation, blindness, ulcers, heart or cardiovascular diseases, nervous system disorders, and susceptibility to infection by pathogenic microorganisms or viruses. Autoimmune diseases include, but are not limited to, rheumatoid arthritis, multiple sclerosis, diabetes, systemic lupus, erythematosus and Grave's disease. Cancers include, but are not limited to, cancers of the bladder, brain, breast, colon, esophagus, kidney, hematopoietic system e.g. leukemia, liver, lung, oral cavity, ovary, pancreas, prostate, skin, stomach, and uterus. A phenotypic trait may also include susceptibility to drug or other therapeutic treatments, appearance, height, color (e.g. of flowering plants), strength, speed (e.g. of race horses), hair color, etc. Many examples of phenotypic traits associated with genetic variation have been described, see e.g., U.S. Pat. No. 5,908,978 (which identifies association of disease resistance in certain species of plants associated with genetic variations) and U.S. Pat. No. 5,942,392 (which describes genetic markers associated with development of Alzheimer's disease).

Identification of associations between genetic variations (e.g. occurrence of SNPs) and phenotypic traits is useful for many purposes. For example, identification of a correlation between the presence of a SNP allele in a subject and the ultimate development by the subject of a disease is particularly useful for administering early treatments, or instituting lifestyle changes (e.g., reducing cholesterol or fatty foods in order to avoid cardiovascular disease in subjects having a greater-than-normal predisposition to such disease), or closely monitoring a patient for development of cancer or other disease. It may also be useful in prenatal screening to identify whether a fetus is afflicted with or is predisposed to develop a serious disease. Additionally, this type of information is useful for screening animals or plants bred for the purpose of enhancing or exhibiting of desired characteristics.

One method for determining an SNP or a plurality of SNPs associated with a plurality of genomes is screening for the presence or absence of a SNP in a plurality of genomic samples derived from organisms with the trait. In order to determine which SNPs are related to a particular phenotypic trait, genomic samples are isolated from a group of individuals which exhibit the particular phenotypic trait, and the samples are analyzed for the presence of common SNPs. The genomic sample obtained from each individual may be combined to form a pooled genomic sample. Then the methods of the invention are used to determine an allelic frequency for each SNP. The pooled genomic sample is screened using panels of SNPs in a high throughput method of the invention to determine whether the presence or absence of a particular SNP (allele) is associated with the phenotype. In some cases, it may be possible to predict the likelihood that a particular subject will exhibit the related phenotype. If a particular polymorphic allele is present in 30% of individuals who develop Alzheimer's disease but only in 1% of the population, then an individual having that allele has a higher likelihood of developing Alzheimer's disease. The likelihood can also depend on several factors such as whether individuals not afflicted with Alzheimer's disease have this allele and whether other factors are associated with the development of Alzheimer's disease. This type of analysis can be useful for determining a probability that a particular phenotype will be exhibited. In order to increase the predictive ability of this type of analysis, multiple SNPs associated with a particular phenotype can be analyzed and the correlation values identified.

It is also possible to identify SNPs which segregate with a particular disease. Multiple polymorphic sites may be detected and examined to identify a physical linkage between them or between a marker (SNP) and a phenotype. This may be used to map a genetic locus linked to or associated with a phenotypic trait to a chromosomal position and thereby revealing one or more genes associated with the phenotypic trait. If two polymorphic sites segregate randomly, then they are either on separate chromosomes or are distant enough, with respect to one another on the same chromosome that they do not co-segregate. If two sites co-segregate with significant frequency, then they are linked to one another on the same chromosome. These types of linkage analyses are useful for developing genetic maps which may define regions of the genome important for a phenotype—including a disease genotype.

Linkage analysis may be performed on family members who exhibit high rates of a particular phenotype or a particular disease. Biological samples are isolated from family members exhibiting a phenotypic trait, as well as from subjects which do not exhibit the phenotypic trait. These samples are each used to generate individual SNPs allelic frequencies. The data can be analyzed to determine whether the various SNPs are associated with the phenotypic trait and whether or not any SNPs segregate with the phenotypic trait.

Methods for analyzing linkage data have been described in many references, including Thompson & Thompson, Genetics in Medicine (5th edition), W.B. Saunders Co., Philadelphia, 1991; and Strachan, “Mapping the Human Genome” in the Human Genome (Bios Scientific Publishers Ltd., Oxford) chapter 4, and summarized in PCT published patent application WO98/18967 by Affymetrix, Inc. Linkage analysis involving by calculating log of the odds values (LOD values) reveals the likelihood of linkage between a marker and a genetic locus at a recombination fraction, compared to the value when the marker and genetic locus are not linked. The recombination fraction indicates the likelihood that markers are linked. Computer programs and mathematical tables have been developed for calculating LOD scores of different recombination fraction values and determining the recombination fraction based on a particular LOD score, respectively. See e.g., Lathrop, PNAS, USA 81, 3443-3446 (1984); Smith et al., Mathematical Tables for Research Workers in Human Genetics (Churchill, London, 1961); Smith, Ann. Hum. Genet. 32, 127-1500 (1968). Use of LOD values for genetic mapping of phenotypic traits is described in PCT published patent application WO98/18967 by Affymetrix, Inc. In general, a positive LOD score value indicates that two genetic loci are linked and a LOD score of +3 or greater is strong evidence that two loci are linked. A negative value suggests that the linkage is less likely.

The methods of the invention are also useful for assessing loss of heterozygosity in a tumor. Loss of heterozygosity in a tumor is useful for determining the status of the tumor, such as whether the tumor is an aggressive, metastatic tumor. The method is can be performed by isolating genomic DNA from tumor sample obtained from a plurality of subjects having tumors of the same type, as well as from normal (i.e., non-cancerous) tissue obtained from the same subjects. These genomic DNA samples are used to for the SNP detection method of the invention. The absence of a SNP allele from the tumor compared to the SNP alleles generated from normal tissue indicates whether loss of heterozygosity has occurred. If a SNP allele is associated with a metastatic state of a cancer, the absence of the SNP allele can be compared to its presence or absence in a non-metastatic tumor sample or a normal tissue sample. A database of SNPs which occur in normal and tumor tissues can be generated and an occurrence of SNPs in a patient's sample can be compared with the database for diagnostic or prognostic purposes.

It is useful to be able to differentiate non-metastatic primary tumors from metastatic tumors, because metastasis is a major cause of treatment failure in cancer patients. If metastasis can be detected early, it can be treated aggressively in order to slow the progression of the disease. Metastasis is a complex process involving detachment of cells from a primary tumor, movement of the cells through the circulation, and eventual colonization of tumor cells at local or distant tissue sites. Additionally, it is desirable to be able to detect a predisposition for development of a particular cancer such that monitoring and early treatment may be initiated. Many cancers and tumors are associated with genetic alterations.

Solid tumors progress from tumorigenesis through a metastatic stage and into a stage at which several genetic aberrations can occur. e.g., Smith et al., Breast Cancer Res. Terat., 18 Suppl. 1, S5-14, 1991. Genetic aberrations are believed to alter the tumor such that it can progress to the next stage, i.e., by conferring proliferative advantages, the ability to develop drug resistance or enhanced angiogenesis, proteolysis, or metastatic capacity. These genetic aberrations are referred to as “loss of heterozygosity.” Loss of heterozygosity can be caused by a deletion or recombination resulting in a genetic mutation which plays a role in tumor progression. Loss of heterozygosity for tumor suppressor genes is believed to play a role in tumor progression. For instance, it is believed that mutations in the retinoblastoma tumor suppressor gene located in chromosome 13q14 causes progression of retinoblastomas, osteosarcomas, small cell lung cancer, and breast cancer. Likewise, the short arm of chromosome 3 has been shown to be associated with cancer such as small cell lung cancer, renal cancer and ovarian cancers. For instance, ulcerative colitis is a disease which is associated with increased risk of cancer presumably involving a multistep progression involving accumulated genetic changes (U.S. Pat. No. 5,814,444). It has been shown that patients afflicted with long duration ulcerative colitis exhibit an increased risk of cancer, and that one early marker is loss of heterozygosity of a region of the distal short arm of chromosome 8. This region is the site of a putative tumor suppressor gene that may also be implicated in prostate and breast cancer. Loss of heterozygosity can easily be detected by performing the methods of the invention routinely on patients afflicted with ulcerative colitis. Similar analyses can be performed using samples obtained from other tumors known or believed to be associated with loss of heterozygosity. The methods of the invention are particularly advantageous for studying loss of heterozygosity because thousands of tumor samples can be screened at one time.

The invention described involves methods for processing nucleic acids to determine an allelic frequency. The method may be broadly defined in the following three steps: (1) Sample preparation—preparation of the first amplicons; (2) bead emulsion PCR—preparation of the second amplicons. (3) sequencing by synthesis—determining multiple sequences from the second amplicons to determine an allelic frequency. Each of these steps is described in more detail below and in the Example section.

### 1. Nucleic Acid Template Preparation

### Nucleic Acid Templates

The template nucleic acid can be constructed from any source of nucleic acid, e.g., any cell, tissue, or organism, and can be generated by any art-recognized method. Alternatively, template libraries can be made by generating a complementary DNA (cDNA) library from RNA, e.g., messenger RNA (mRNA). Methods of sample preparation may be found in copending U.S. application Ser. No. 10/767,779 and PCT application US04/02570 and is also published in WO/04070007—all incorporated herein by reference in their entirety.

One preferred method of nucleic acid template preparation is to perform PCR on a sample to amplify a region containing the allele or alleles of interest. The PCR technique can be applied to any nucleic acid sample (DNA, RNA, cDNA) using oligonucleotide primers spaced apart from each other. The primers are complementary to opposite strands of a double stranded DNA molecule and are typically separated by from about 50 to 450 nucleotides or more (usually not more than 2000 nucleotides). The PCR method is described in a number of publications, including Saiki et al., Science (1985) 230:1350-1354; Saiki et al., Nature (1986) 324:163-166; and Scharf et al., Science (1986) 233:1076-1078. Also see U.S. Pat. Nos. 4,683,194; 4,683,195; and 4,683,202, the text of each patent is herein incorporated by reference. Additional methods for PCR amplification are described in: PCR Technology: Principles and Applications for DNA Amplification ed. HA Erlich, Freeman Press, New York, N.Y. (1992); PCR Protocols: A Guide to Methods and Applications, eds. Innis, Gelfland, Snisky, and White, Academic Press, San Diego, Calif. (1990); Mattila et al. (1991) Nucleic Acids Res. 19: 4967; Eckert, K. A. and Kunkel, T. A. (1991) PCR Methods and Applications 1: 17, and; PCR, eds. McPherson, Quirkes, and Taylor, IRL Press, Oxford, which are incorporated herein by reference.

### 2. Nucleic Acid Template Amplification

In order for the nucleic acid template (i.e., the amplicons generated by the PCR method of the first step) to be sequenced according to the methods of this invention the copy number must be amplified a second time to generate a sufficient number of copies of each template to produce a detectable signal by the light detection means. Any suitable nucleic acid amplification means may be used. In a preferred embodiment, a novel amplification system, herein termed EBCA (Emulsion Based Clonal Amplification or bead emulsion amplification) is used to perform this second amplification.

EBCA is performed by attaching a template nucleic acid (e.g., DNA) to be amplified to a solid support, preferably in the form of a generally spherical bead. A library of single stranded template DNA prepared according to the sample preparation methods of this invention is an example of one suitable source of the starting nucleic acid template library to be attached to a bead for use in this amplification method.

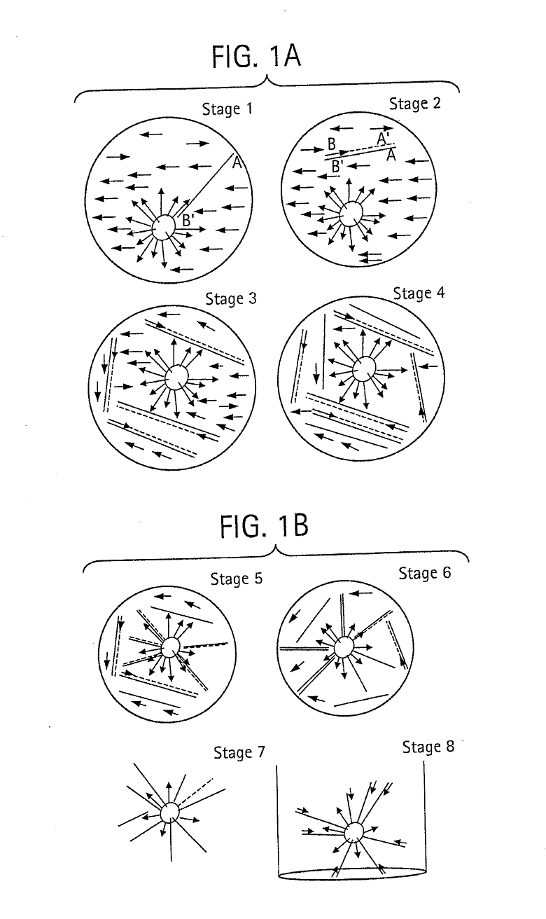

The bead is linked to a large number of a single primer species (i.e., primer B in FIG. 1) that is complementary to a region of the template DNA. Template DNA annealed to the bead bound primer. The beads are suspended in aqueous reaction mixture and then encapsulated in a water-in-oil emulsion. The emulsion is composed of discrete aqueous phase microdroplets, approximately 60 to 200 um in diameter, enclosed by a thermostable oil phase. Each microdroplet contains, preferably, amplification reaction solution (i.e., the reagents necessary for nucleic acid amplification). An example of an amplification would be a PCR reaction mix (polymerase, salts, dNTPs) and a pair of PCR primers (primer A and primer B). See, FIG. 1A. A subset of the microdroplet population also contains the DNA bead comprising the DNA template. This subset of microdroplet is the basis for the amplification. The microcapsules that are not within this subset have no template DNA and will not participate in amplification. In one embodiment, the amplification technique is PCR and the PCR primers are present in a 8:1 or 16:1 ratio (i.e., 8 or 16 of one primer to 1 of the second primer) to perform asymmetric PCR.

In this overview, the DNA is annealed to an oligonucleotide (primer B) which is immobilized to a bead. During thermocycling (FIG. 1B), the bond between the single stranded DNA template and the immobilized B primer on the bead is broken, releasing the template into the surrounding microencapsulated solution. The amplification solution, in this case, the PCR solution, contains addition solution phase primer A and primer B. Solution phase B primers readily bind to the complementary b′ region of the template as binding kinetics are more rapid for solution phase primers than for immobilized primers. In early phase PCR, both A and B strands amplify equally well (FIG. 1C).

By midphase PCR (i.e., between cycles 10 and 30) the B primers are depleted, halting exponential amplification. The reaction then enters asymmetric amplification and the amplicon population becomes dominated by A strands (FIG. 1D). In late phase PCR (FIG. 1E), after 30 to 40 cycles, asymmetric amplification increases the concentration of A strands in solution. Excess A strands begin to anneal to bead immobilized B primers. Thermostable polymerases then utilize the A strand as a template to synthesize an immobilized, bead bound B strand of the amplicon.

In final phase PCR (FIG. 1F), continued thermal cycling forces additional annealing to bead bound primers. Solution phase amplification may be minimal at this stage but concentration of immobilized B strands increase. Then, the emulsion is broken and the immobilized product is rendered single stranded by denaturing (by heat, pH etc.) which removes the complimentary A strand. The A primers are annealed to the A′ region of immobilized strand, and immobilized strand is loaded with sequencing enzymes, and any necessary accessory proteins. The beads are then sequenced using recognized pyrophosphate techniques (described, e.g., in U.S. Pat. Nos. 6,274,320, 6258,568 and 6,210,891, incorporated in toto herein by reference).

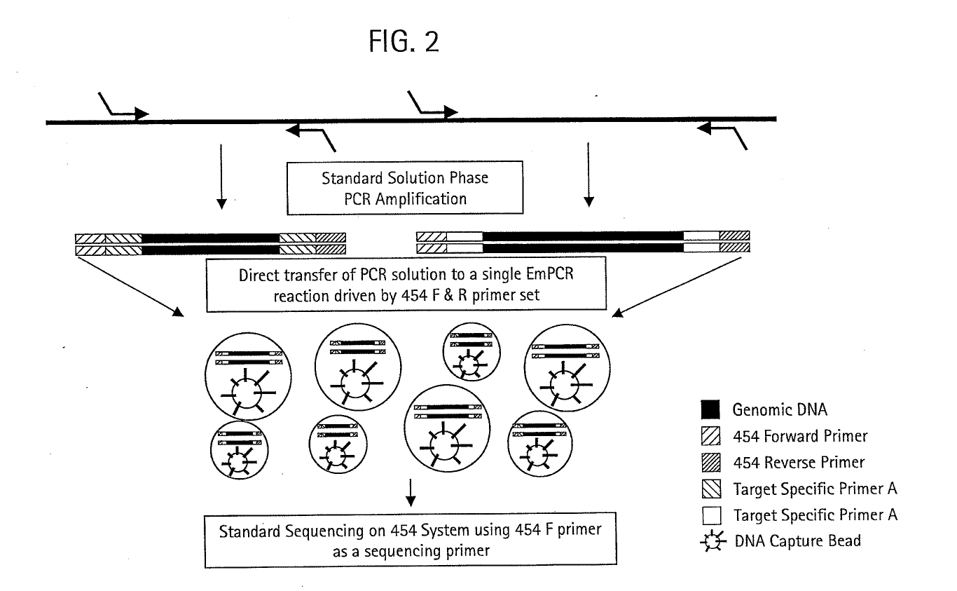

In a preferred embodiment, the primers used for amplification are bipartite—comprising a 5′ section and a 3′ section. The 3′ section of the primer contains target specific sequence (see FIG. 2) and performed the function of PCR primers. The 5′ section of the primer comprises sequences which are useful for the sequencing method or the immobilization method. For example, in FIG. 2, the 5′ section of the two primers used for amplification contains sequences (labeled 454 forward and 454 reverse) which are complementary to primers on a bead or a sequencing primer. That is, the 5′ section, containing the forward or reverse sequence, allows the amplicons to attach to beads that contain immobilized oligos which are complementary to the forward or reverse sequence. Furthermore, sequencing reaction may be initiated using sequencing primers which are complementary to the forward and reverse primer sequences. Thus one set of beads comprising sequences complementary to the 5′ section of the bipartite primer may be used on all reactions. Similarly, one set of sequencing primers comprising sequences complementary to the 5′ section of the bipartite primer may be used to sequence any amplicons made using the bipartite primer. In the most preferred embodiment, all bipartite primer sets used for amplification would have the same set of 5′ sections such as the 454 forward primer and 454 reverse primer shown in FIG. 2. In this case, all amplicons may be analyzed using standard beads coated with oligos complementary to the 5′ section. The same oligos (immobilized on beads or not immobilized) may be used as sequencing oligos.

### Breaking the Emulsion and Bead Recovery

Following amplification of the template, the emulsion is “broken” (also referred to as “demulsification” in the art). There are many methods of breaking an emulsion (see, e.g., U.S. Pat. No. 5,989,892 and references cited therein) and one of skill in the art would be able to select the proper method. One preferred method of breaking the emulsion is described in detail in the Example section.

After the emulsion is broken, the amplified template-containing beads may then be resuspended in aqueous solution for use, for example, in a sequencing reaction according to known technologies. (See, Sanger, F. et al., Proc. Natl. Acad. Sci. U.S.A. 75, 5463-5467 (1977); Maxam, A. M. & Gilbert, W. Proc Natl Acad Sci USA 74, 560-564 (1977); Ronaghi, M. et al., Science 281, 363, 365 (1998); Lysov, I. et al., Dokl Akad Nauk SSSR 303, 1508-1511 (1988); Bains W. & Smith G. C. J. TheorBiol 135, 303-307 (1988); Drnanac, R. et al., Genomics 4, 114-128 (1989); Khrapko, K. R. et al., FEBS Lett 256. 118-122 (1989); Pevzner P. A. J Biomol Struct Dyn 7, 63-73 (1989); Southern, E. M. et al., Genomics 13, 1008-1017 (1992).) If the beads are to be used in a pyrophosphate-based sequencing reaction (described, e.g., in U.S. Pat. No. 6,274,320, 6,258,568 and 6,210,891, and incorporated in toto herein by reference), then it is necessary to remove the second strand of the PCR product and anneal a sequencing primer to the single stranded template that is bound to the bead.

At this point, the amplified DNA on the bead may be sequenced either directly on the bead or in a different reaction vessel. In an embodiment of the present invention, the DNA is sequenced directly on the bead by transferring the bead to a reaction vessel and subjecting the DNA to a sequencing reaction (e.g., pyrophosphate or Sanger sequencing). Alternatively, the beads may be isolated and the DNA may be removed from each bead and sequenced. In either case, the sequencing steps may be performed on each individual bead.

### 3. Methods of Sequencing Nucleic Acids

One method of sequencing is pyrophosphate-based sequencing. In pyrophosphate based sequencing sample DNA sequence and the extension primer subjected to a polymerase reaction in the presence of a nucleotide triphosphate whereby the nucleotide triphosphate will only become incorporated and release pyrophosphate (PPi) if it is complementary to the base in the target position, the nucleotide triphosphate being added either to separate aliquots of sample-primer mixture or successively to the same sample-primer mixture. The release of PPi is then detected to indicate which nucleotide is incorporated.

In an embodiment, a region of the sequence product is determined by annealing a sequencing primer to a region of the template nucleic acid, and then contacting the sequencing primer with a DNA polymerase and a known nucleotide triphosphate, i.e., dATP, dCTP, dGTP, dTTP, or an analog of one of these nucleotides. The sequence can be determined by detecting a sequence reaction byproduct, as is described below.

The sequence primer can be any length or base composition, as long as it is capable of specifically annealing to a region of the amplified nucleic acid template. No particular structure for the sequencing primer is required so long as it is able to specifically prime a region on the amplified template nucleic acid. Preferably, the sequencing primer is complementary to a region of the template that is between the sequence to be characterized and the sequence hybridizable to the anchor primer. The sequencing primer is extended with the DNA polymerase to form a sequence product. The extension is performed in the presence of one or more types of nucleotide triphosphates, and if desired, auxiliary binding proteins.

Incorporation of the dNTP is preferably determined by assaying for the presence of a sequencing byproduct. In a preferred embodiment, the nucleotide sequence of the sequencing product is determined by measuring inorganic pyrophosphate (PPi) liberated from a nucleotide triphosphate (dNTP) as the dNMP is incorporated into an extended sequence primer. This method of sequencing, termed Pyrosequencing™ technology (PyroSequencing AB, Stockholm, Sweden) can be performed in solution (liquid phase) or as a solid phase technique. PPi-based sequencing methods are described generally in, e.g., WO9813523A1, Ronaghi, et al., 1996. Anal. Biochem. 242: 84-89, Ronaghi, et al., 1998. Science 281: 363-365 (1998) and USSN 2001/0024790. These disclosures of PPi sequencing are incorporated herein in their entirety, by reference. See also, e.g., U.S. Pat. Nos. 6,210,891 and 6,258,568, each fully incorporated herein by reference.

In a preferred embodiment, DNA sequencing is performed using 454 corporation's (454 Life Sciences) sequencing apparatus and methods which are disclosed in copending patent applications USSN: 10/768,729, USSN: 10/767,779, USSN: 10/767,899, and USSN: 10/767,894—all of which are filed Jan. 28, 2004.

Unless otherwise defined, all technical and scientific terms used herein have the same meaning as commonly understood by one of ordinary skill in the art to which this invention belongs. Commonly understood definition would include those defined in USSN: 60/476,602, filed Jun. 6, 2003; USSN: 60/476,504, filed Jun. 6, 2003; USSN: 60/443,471, filed Jan. 29, 2003; USSN: 60/476,313, filed Jun. 6, 2003; USSN: 60/476,592, filed Jun. 6, 2003; USSN: 60/465,071, filed Apr. 23, 2003; USSN: 60/497,985, filed Aug. 25, 2003; USSN: 10/767,779 filed Jan. 28, 2004; 10/767,899 filed. Jan. 28, 2004; USSN: 10/767,894 filed Jan. 28, 2004. All patents, patent applications, and references cited in this application are hereby fully incorporated by reference.

### EXAMPLES

### Example 1

### Sequencing of the HLA Locus

Five PCR primer pairs were designed to span known, publicly disclosed SNPs in the MHC class II locus. Primers were design using the Primer3 software (Whitehead Institute for Biomedical Research) using approx. 200 base-pair long genomic sequences encompassing the target regions as input. Each primer consisted of a locus specific 3′ portion ranging in length from 20 to 24 bases and a constant 19 base 5′ portion (shown in lowercase) that includes a 4 base key (high-lighted in bold). Primers were purchased from Integrated DNA Technologies (Coralville, Iowa):

SAD1F-DC1
(SEQ ID NO: 1)
gcctccctcgcgcca tcag ACCTCCCTCTGTGTCCTTACAA
SAD1R-DC1
(SEQ ID NO: 2)
gccttgccagcccgc tcag GGAGGGAATCATACTAGCACCA
SAD1F-DD14
(SEQ ID NO: 3)
gcctccctcgcgcca tcag TCTGACGATCTCTGTCTTCTAACC
SAD1R-DD14
(SEQ ID NO: 4)
gccttgccagcccgc tcag GCCTTGAACTACACGTGGCT
SAD1F-DE15
(SEQ ID NO: 5)
gcctccctcgcgcca tcag ATTTCTCTACCACCCCTGGC
SAD1R-DE15
(SEQ ID NO: 6)
gccttgccagcccgc tcag AGCTCATGTCTCCCGAAGAA
SAD1F-GA9
(SEQ ID NO: 7)
gcctccctcgcgcca tcag AAAGCCAGAAGAGGAAAGGC
SAD1R-GA9
(SEQ ID NO: 8)
gccttgccagcccgc tcag CTTGCAGATTGGTCATAAGG
SAD1F-F5
(SEQ ID NO: 9)
gcctccctcgcgcca tcag ACAGTGCAAACACCACCAAA
SAD1R-F5
(SEQ ID NO: 10)
gccttgccagcccgc tcag CCAGTATTCATGGCAGGGTT

Human genomic DNA (Cornell Medical Institute for Research, Camden, N.J.) from 4 individuals was quantitated based on optical density at 260 nm and 100 ng (approx. 15,000 haploid genome equivalents) was used as template for each PCR amplification reaction. PCR reactions were performed under standard reaction conditions (60 mM Tris-SO₄, pH 8.9, 18 mM (NH₄)₂SO₄), 2.5 mM MgSO₄, 1 mM dNTPs, 0.625 uM of each primer, 4.5 units Platinum Tag High Fidelity polymerase (Invitrogen, Carlsbad, Calif.)) with the following temperature profile: 3 mM 94° C.; 30 cycles of 30 s 94° C., 45 s 57° C., 1 min 72° C.; 3 min 72° C. Amplification products were purified using a QiaQuick PCR Purification kit (Qiagen, Valencia, Calif.), and their anticipated sizes (156 to 181 base pairs) were verified on a 2100 BioAnalyzer microfluidics instrument using a 500 DNA LabChip® (Agilent Technologies, Inc, Palo Alto, Calif.). The purified amplicons were quantitated with a PicoGreen® dsDNA quantitation kit (Molecular Probes, Eugene, Oreg.) and diluted to 10⁷ copies per microliter.

EBCA (Emulsion Based Clonal Amplification) was performed as described above with 0.5 amplicons per bead, using amplification primers SAD1F (GCC TCC CTC GCG CCA (SEQ ID NO:11)) and SAD1R and Sepharose capture beads with SADR1 (GCC TTG CCA GCC CGC (SEQ ID NO:12)) capture primer (Amersham BioSciences, Piscataway, N.J.). All further manipulations, including breaking of the emulsions and sequencing on the PicoTiter plate were performed as described above.

### Example 2

### Sensitive Mutation Detection

To demonstrate the capability of the current system (i.e., the 454 platform) to detect low abundance sequence variants, specifically single base substitutions, experiments were designed to sequence known alleles mixed at various ratios.

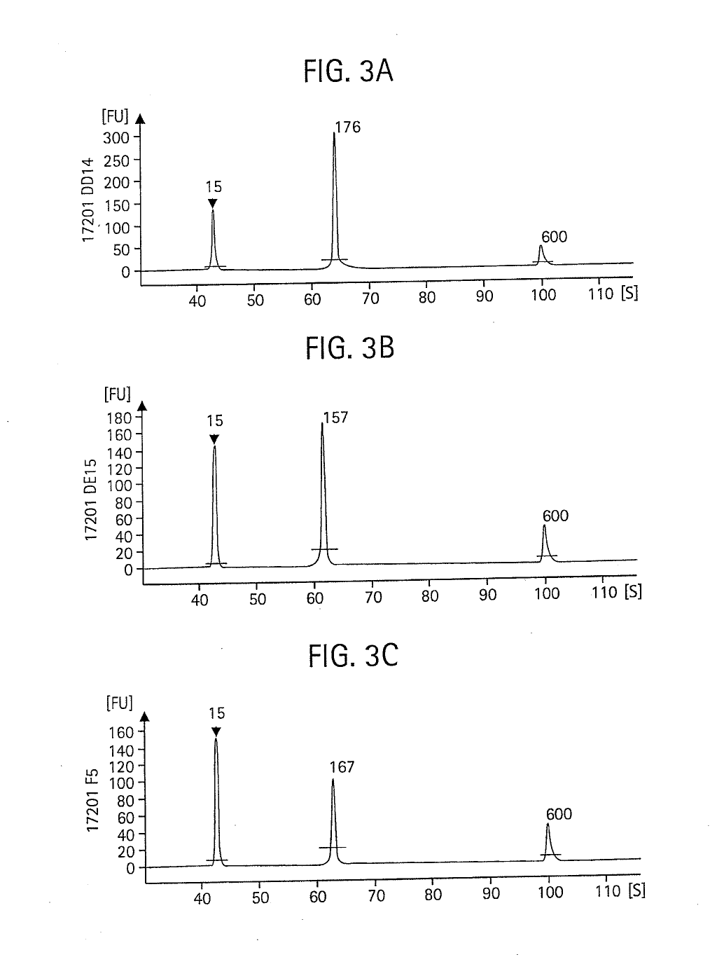

The 6 primer pairs listed above were tested for amplification efficiency and further analysis was performed using pairs SAD1F/R-DD14, SAD1F/R-DE15 and SAD1F/R-F5 which all produced distinct amplification products (FIG. 3). A total of 8 human genomic DNA samples were amplified and sequenced on the 454 platform to determine the genotypes for each locus. To simplify the experimental setup all further analysis was done using primer pair SAD1F/R-DD14 (FIG. 3A) and two samples shown to be homozygous for either the C or T allele at the particular locus.

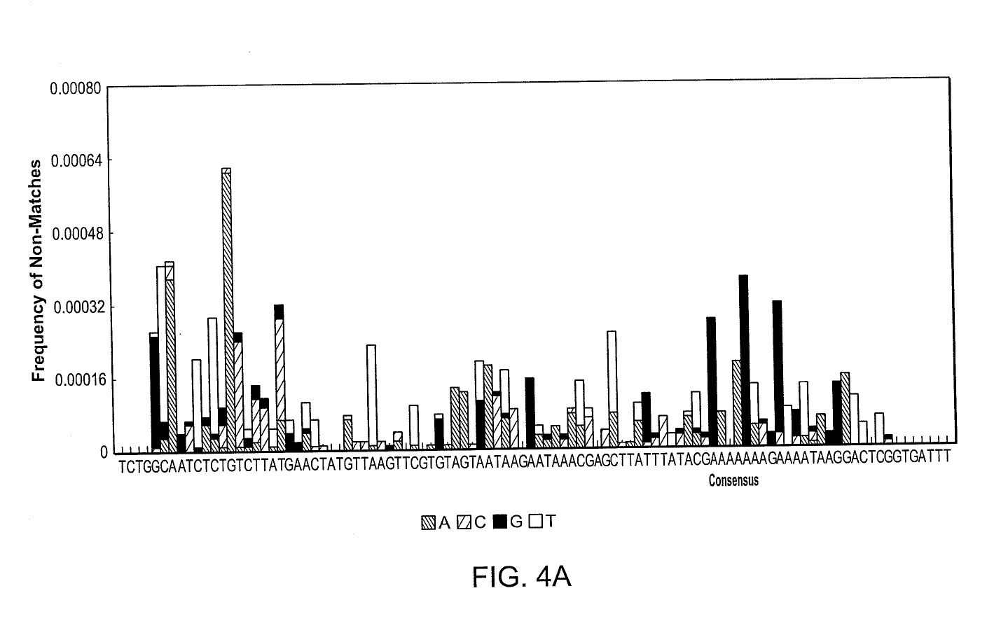

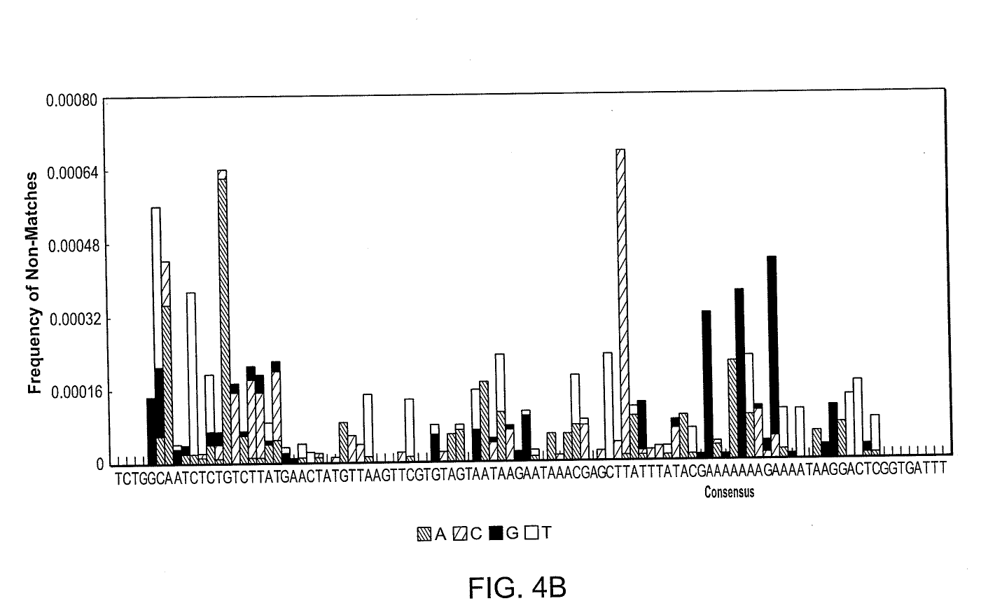

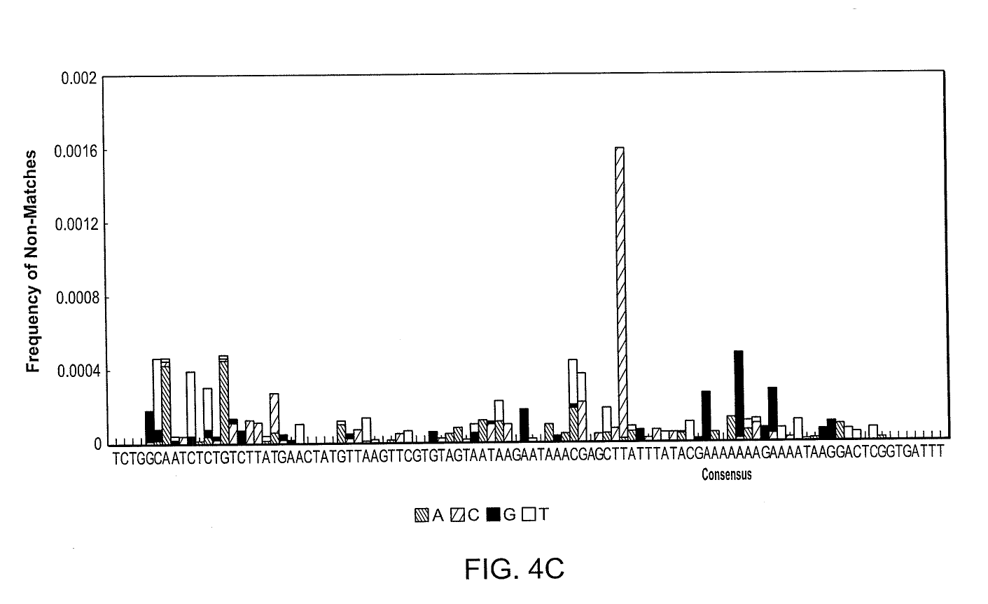

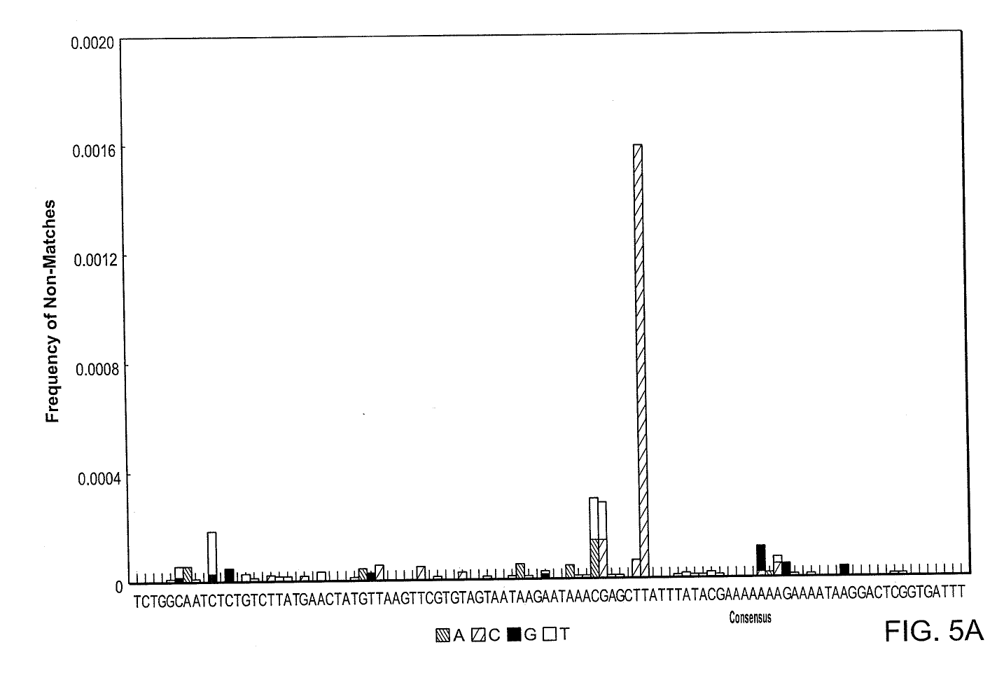

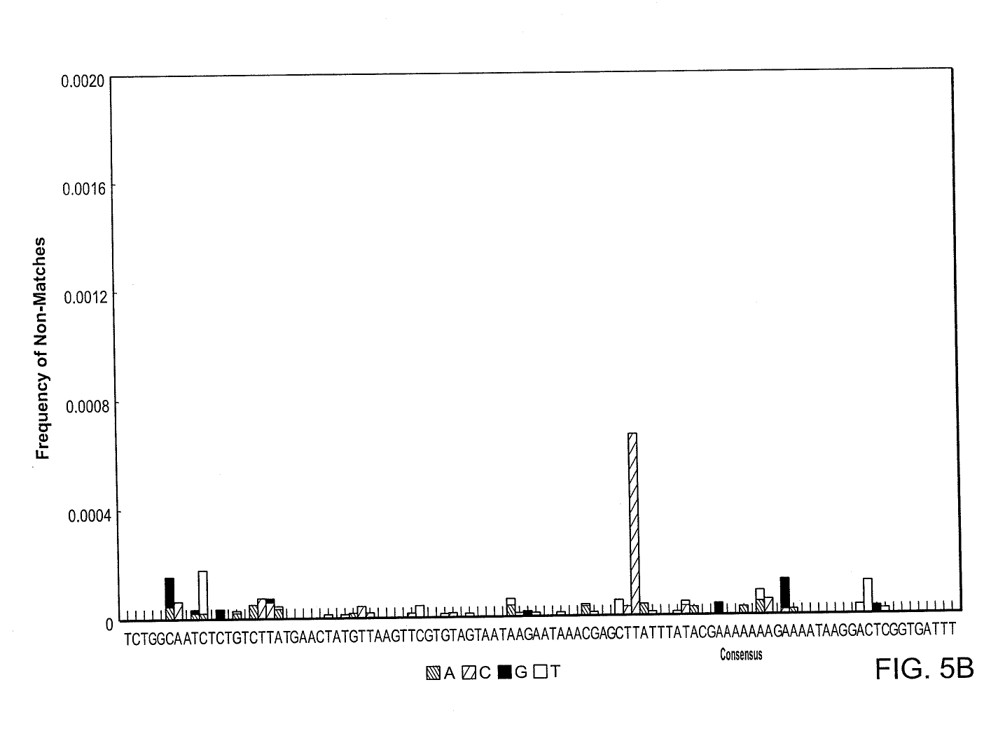

The primary amplicons from each sample were quantitated and mixed at specific ratios ranging from 10:90 down to 1:1000, typically with the T allele in excess. After mixing the samples were diluted to a working concentration of 2×10⁶ copies per microliter and subjected to EBCA and sequenced on the 454 platform. FIG. 2 presents sequencing data obtained from the mixing of the C allele in approximate ratios 1:500 and 1:1000 into the T allele. In both cases roughly 10,000 high-quality sequencing reads were generated and subjected to Blast analysis to identify nucleotide substitutions against a reference sequence (in this case the T allele carrying sequence). For visualization of the results the substitution frequency is plotted in a color-coded fashion relative to the reference sequence. The data demonstrate that in both samples the low frequency single base substitutions were readily identified (FIG. 4A-C). Furthermore the background was found to be relatively consistent between samples allowing background subtraction. This typically produced a signal-to-noise ratio even for the 1:1000 allele that exceeded 10 (FIGS. 5A and B). Additional experimentation using samples of known genotypes has confirmed the ability to detect single nucleotide substitutions down to at least a 0.1% abundance level. Additional confidence in low abundance changes can be obtained from sequencing a template in both directions. Typically the difference between the frequencies from the two independent bidirectional data sets is within 20% down to the 1% abundance level.

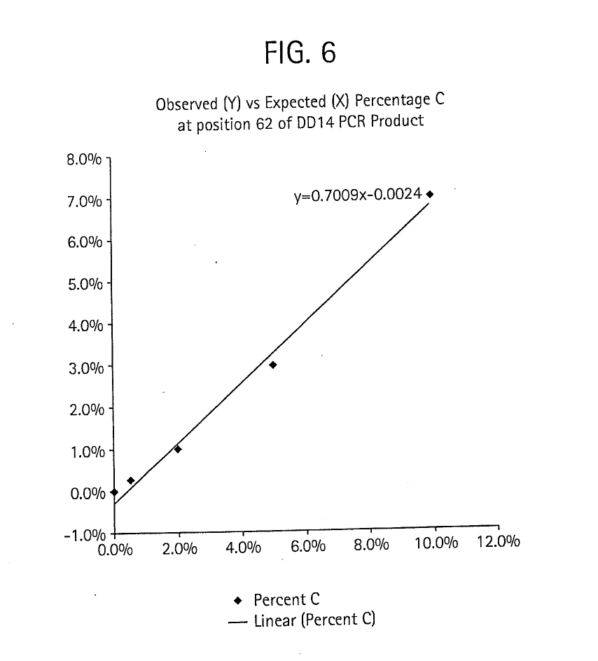

To demonstrate a linear response over a broader range of allelic ratios, amplicons representing the T and C alleles from the DD14 HLA locus were mixed in ratios 1:10, 1:20, 1:50 and 1:200 (10%, 5%, 2% and 0.5%), EBCA amplified and sequenced. FIG. 6 shows that a linear increase in the relative number of low frequency allele was observed throughout the range (R²=0.9927). The recorded absolute frequencies somewhat deviated from the intended ratios (See Table below) and were attributed to commonly observed difficulties trying to precisely quantitate, aliquot and mix small amounts of DNA.

| Expected | Total | Expected | Observed | Observed | Observed |
|---|---|---|---|---|---|
| Percent C | Reads | C | C | T | Percent C |
| 0.00% | 101450 | 0 | 1 | 101449 | 0.00% |
| 0.50% | 72406 | 361 | 193 | 72213 | 0.27% |
| 2.00% | 103292 | 2045 | 1049 | 102243 | 1.02% |
| 2.00% | 57115 | 1131 | 578 | 56537 | 1.01% |
| 5.00% | 112378 | 5452 | 3340 | 109038 | 2.97% |
| 10.00% | 104906 | 9760 | 7311 | 97595 | 6.97% |

Summary of sequencing used to generate plot in FIG. 6.
Numbers in columns 2-5 indicate total number of sequenced templates, and the expected and observed numbers for each allele respectively.

### Example 3

### Bacterial 16S Project—A Method to Examine Bacteria Populations

Bacterial population surveys are essential applications for many fields including industrial process control, in addition to medical, environmental and agricultural research. One common method utilizes the 16S ribosomal RNA gene sequence to distinguish bacterial species (Jonasson, Olofsson et al. 2002; Grahn, Olofsson et al. 2003). Another method similarly examines the intervening sequence between the 16S and 23S ribosomal RNA genes (Garcia-Martinez, Bescos et al. 2001). However, the majority of researchers find a complete census of complex bacterial populations is impossible using current sample preparation and sequencing technologies; the labor requirements for such a project are either prohibitively expensive or force dramatic subsampling of the populations.

Currently, high throughput methods are not routinely used to examine bacterial populations. Common practice utilizes universal primer(s) to amplify the 16S ribosomal RNA gene (or regions within the gene), which are subsequently subcloned into vectors and sequenced. Restriction digests are often conducted on the vectors in an effort to reduce the sequencing load by eliminating vectors which exhibit identical restriction patterns. Resultant sequences are compared to a database of known genes from various organisms; estimates of population composition are drawn from the presence of species- or genus-specific gene sequences. The methods of this disclosure has the potential to revolutionize the study of bacterial populations by drastically reducing the labor costs through eliminating cloning and restriction digest steps, increasing informational output by providing complete sequences from the 16S (and possibly intergenic and 23S) RNA regions possibly allowing previously unobtainable substrain differentiation, and potentially providing estimates of species density by converting sequence oversampling into relative abundance.

One preferred method of nucleic acid sequencing is the pyrophosphate based sequencing methods developed by 454 Life Sciences. Utilization of the methods of the invention coupled with all aspects of the massively parallel 454 technology (some of which is disclosed in this specification) can greatly increase the throughput and reduce the cost of community identification. The 454 technology eliminates the need to clone large numbers of individual PCR products while the small size of the 16S gene (1.4 kb) allows tens of thousands of samples to be processed simultaneously. The process has been successfully demonstrated in the manner outlined below.

Initially, Escherichia coli 16S DNA was obtained from E. coli TOP10 competent cells (Invitrogen, Carlsbad, Calif.) transformed with the PCR2.1 vector, plated onto LB/Ampicillin plates (50 μg/ml) and incubated overnight at 37° C. A single colony was picked and inoculated into 3 ml of LB/Ampicillin broth and shaken at 250 RPM for 6 hours at 37° C. One microliter of this solution was used as template for amplifying the V1 and V3 regions of the 16S sequence.

Bipartite PCR primers were designed for two variable regions in the 16S gene, denoted V1 and V3 as described in Monstein et al (Monstein, Nikpour-Badr et al. 2001). Five prime tags comprised of 454 specific, 19 base (15 base amplification primers, followed by a 3′, 4 base (TCGA) key) forward or reverse primers were fused to the region specific forward and reverse primers that flank the variable V1 and V3 regions. This may be represented as: 5′-(15 base forward or reverse Amplification primer)-(4 base key)-(forward or reverse V1 or V3 primer)-3′. The primers used to produce 16S amplicons contain the following sequences, with the sequences in capital letter representing the V1 or V3 specific primers, the four bases in bold identify the key, and the lower case bases indicate the 454 amplification primers:

SAD-V1 fusion (forward):
(SEQ ID NO: 13)
gcctccctcgcgcca tcag GAAGAGTTTGATCATGGCTCAG
SAD-V1 fusion (reverse):
(SEQ ID NO: 14)
gccttgccagcccgc tcag TTACTCACCCGTCCGCCACT
SAD-V3 fusion (forward):
(SEQ ID NO: 15)
gcctccctcgcgcca tcag GCAACGCGAAGAACCTTACC
SAD-V3 fusion (reverse):
(SEQ ID NO: 16)
gccttgccagcccgc tcag ACGACAGCCATGCAGCACCT

The V1 and V3 amplicons were generated separately in PCR reactions that contained the following reagents: 1× HiFi buffer, 2.5 mM MgSO₄ (Invitrogen), 1 mM dNTPs (Pierce, Milwaukee Wis.), 1 μM each forward and reverse bipartite primer for either V1 or V3 regions (IDT, Coralville, Iowa), 0.15 U/μl Platinum HiFi Taq (Invitrogen). One microliter of E. coli/LB/Ampicillin broth was added to the reaction mixture and 35 cycles of PCR were performed (94° C. for 30 seconds, 55° C. for 30 seconds, and 68° C. for 150 seconds, with the final cycle followed by a 10° C. infinite hold). Subsequently, 1 μl of the amplified reaction mix was run on the Agilent 2100 Bioanalyzer (Agilent, Palo Alto, Calif.) to estimate the concentration of the final product, and assure the proper size product 155 bp for the V1, 145 bp for the V3) was generated.

The V1 and V3 products were then combined, emulsified at template concentrations ranging from 0.5 to 10 template molecules per DNA capture bead and amplified through the EBCA (Emulsion Based Clonal Amplification) process as outlined in the EBCA Protocol section below. The resulting clonally amplified beads were subsequently sequenced on the 454 Genome Sequencer (454 Life Sciences, Branford CT).

The sequences obtained from the amplified beads were aligned against the Escherichia coli 16S gene sequence (Entrez gi174375). Acceptable (or “mapped”) alignments were distinguished from rejected (or “unmapped”) alignments by calculating the alignment score for each sequence. The score is the average logarithm of the probability that an observed signal corresponds to the expected homopolymer, or:

S=Σ ln [P(s|h)]/N

where S is the computed alignment score, P is the probability at a specific flow, s is the signal measured at that flow, h is the length of the reference homopolymer expected at that flow, and N is the total number of flows aligned. The alignment score for each sequence was then compared to a Maximum Alignment Score, or MAS; alignments scoring less than the MAS were considered “real” and were printed to the output file. For this project, a MAS of 1.0 (roughly equivalent to 95% identity) was used.

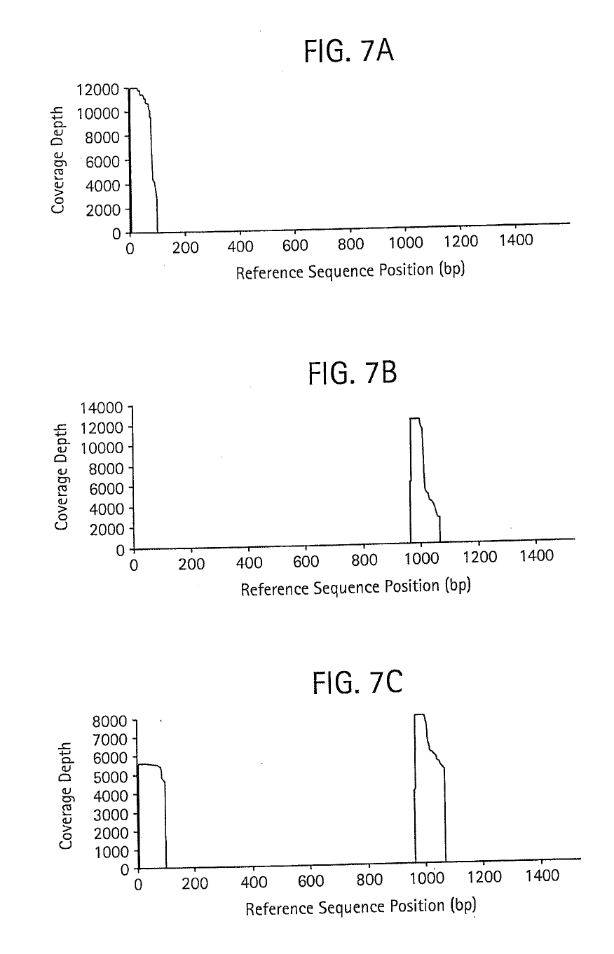

For the sequences generated with the V1 specific primers, of the 13702 sequences generated, 87.75% or 11973 reads mapped to the genome with an alignment score less than 1.0, and a read length greater than 21 bases. A graphical display showing the location of the reads mapping to the 1.6 Kb 16S gene fragment is shown in FIG. 7A, indicating roughly 12,000 reads mapping to the first 100 bases of the 16S gene.

BLASTing the unmodified consensus sequence (AAGAGTTTtGATCATGGCTCAGATTGAACGCTGGCGGCAGGCCTAACACATG CAAGTCGA ACGGTAACAGGA (SEQ ID NO:17)) against the 16S database (http://greengenes.llnl.gov) matched Escherichia coli as the first known organism

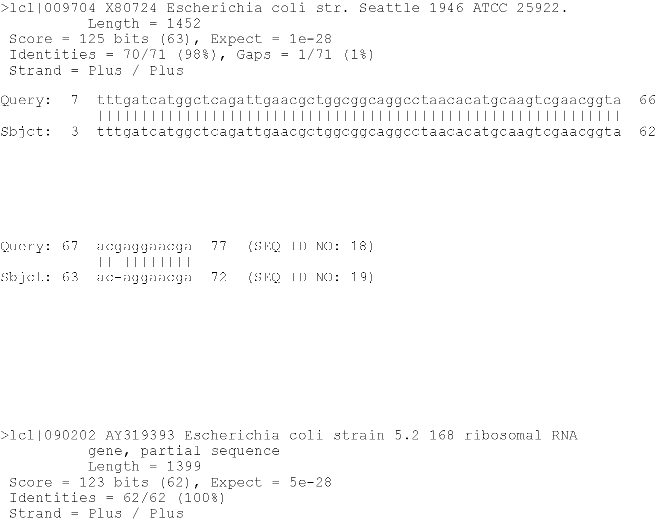

The V1 consensus sequence was edited to AAGAGTTTTGATCATGGCTCAGATTGAACGCTGGCGGCAGGCCTAACACATG CAAGTCGAACGGTAACAGGA (SEQ ID NO:20), as the fourth “T” at position 9 (marked in bold and underline) of a homoploymer stretch was reviewed and removed, based on an exceedingly low confidence score. The BLAST results of the edited V1 sequence demonstrated improved hits against Escherichia coli 16S genes.

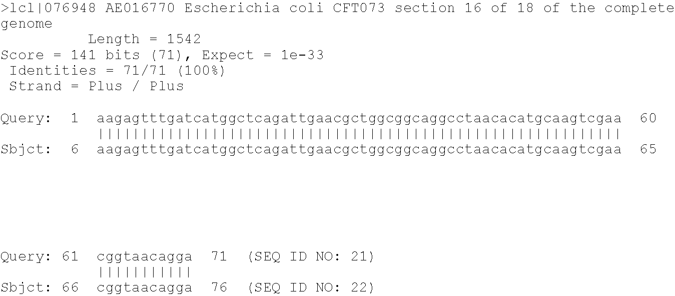

Similar results were obtained with the V3 specific primers. Of the 17329 reads, 71.00% mapped to the 16S reference genome under identical analysis conditions as used with the V1 templates above. This is a lower number than the 87.75% of V1 reads that mapped, and this may reveal a greater diverge between the V3 sample and reference sequences than between the V1 sample and reference sequences. The consensus sequence: CAACGCGAAGAACCTTACCTGGTCTTGACATCCACGAAGTTTACTAGAGATG AGAATGTGCCGTTCGGGAACCGGTGAGACAGGTGCTGCATGGCTGTCGTCTg (SEQ ID NO:23), mapped to regions 966-1067 of the reference genome as shown in FIG. 7B.

Unlike the V1 sequence BLAST results from the unmodified consensus sequence did not match Escherichia coli as the first known organism, but rather as the second organism.

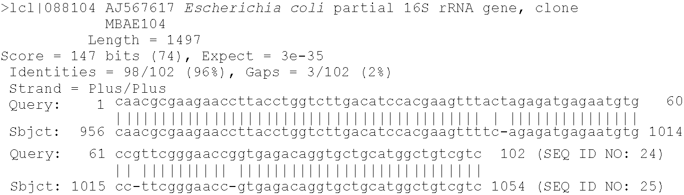

The consensus sequence was reviewed and edited to CAACGCGAAGAACCTTACCTGGTCTTGACATCCACGAAGTTTACAGAGATGA GAATGTGCCGTTCGGGAACCGTGAGACAGGTGCTGCATGGCTGTCGTCTg (SEQ ID NO:26)(with the removal of two bases) based on the confidence scores, and reBLASTed. The BLAST resulted in the highest ranked hit occurring against E. coli.

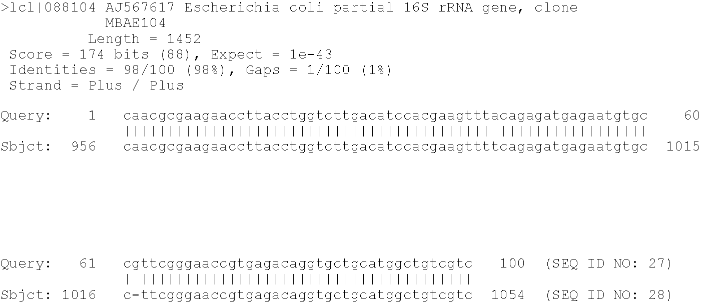

A second experiment was conducted to demonstrate the ability to use mixed PCR primers on unprocessed bacterial cells, where the E. coli cells were grown to saturation and 1 μl of a 1:1000 dilution of the bacterial broth was added to the EBCA reaction mix in lieu of template. The primers used in the EBCA reaction consisted of V1- and V3-specific bipartite primers at 0.04 μM each, as well as the forward and reverse 454 amplification primers at 0.625 and 0.04 μM respectively. Otherwise, the EBCA protocol outlined below was followed.

The data showed that V1 and V3 regions could be successfully amplified, sequenced and distinguished simultaneously from an untreated pool of bacterial cells. Of the 15484 reads, 87.66% mapped to the 16S reference genome, with the sequences located at the distinctive V1 and V3 positions shown in FIG. 7C.

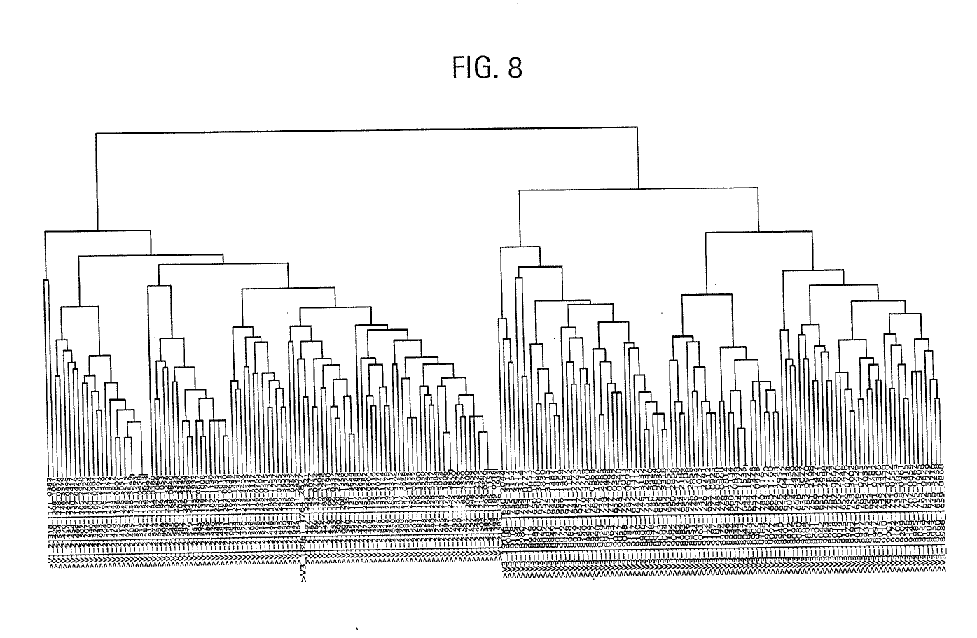

The ability to distinguish between V1 and V3 sequences was assessed by pooling 100 reads of both V1 and V3 sequences, and converting the raw signal data into a binary string, with a “1” indicating that a base was present at a given flow, and a “0” indicating that it was absent. Homopolymer stretches were collapsed into a single positive value, so that “A”, “AA”, and “AAAAA” (SEQ ID NO:29) all received an identical score of “1”. The collapsed binary strings were then clustered via the Hierarchical Ordered Partitioning and Collapsing Hybrid (HOPACH) methodology (Pollard and van der Laan 2005) in the R statistical package (Team 2004). The resulting phylogentic tree, shown in FIG. 8, clearly discriminates between the V1 (shorter length red labels) and the V3 (longer length blue labels) sequences in all but 1 of the 200 sequences.

The ability to discriminate this clearly between two similar regions from the same gene within the same organism suggest that this technology should prove adept at discriminating between variable regions from distinct organisms, providing a valuable diagnostic tool.

### Example 4

### EBCA Protocol

### 4.1 Preparation of DNA Capture Beads

Packed beads from a 1 mL N-hydroxysuccinimide ester (NHS)-activated Sepharose HP affinity column (Amersham Biosciences, Piscataway, N.J.) were removed from the column and activated as described in the product literature (Amersham Pharmacia Protocol #71700600AP). Twenty-five microliters of a 1 mM amine-labeled HEG capture primer (5′-Amine-3 sequential 18-atom hexa-ethyleneglycol spacers CCATCTGTTGCGTGCGTGTC-3′ (SEQ ID NO:30)) (IDT Technologies, Coralville, Iowa, USA) in 20 mM phosphate buffer, pH 8.0, were bound to the beads, after which 25-36 μm beads were selected by serial passage through 36 and 25 μm pore filter mesh sections (Sefar America, Depew, N.Y., USA). DNA capture beads that passed through the first filter, but were retained by the second were collected in bead storage buffer (50 mM Tris, 0.02% Tween, 0.02% sodium azide, pH 8), quantitated with a Multisizer 3 Coulter Counter (Beckman Coulter, Fullerton, Calif., USA) and stored at 4° C. until needed.

### 4.2 Binding Template Species to DNA Capture Beads

Template molecules were annealed to complementary primers on the DNA Capture beads in a UV-treated laminar flow hood. Six hundred thousand DNA capture beads suspended in bead storage buffer were transferred to a 200 μL PCR tube, centrifuged in a benchtop mini centrifuge for 10 seconds, the tube rotated 180° and spun for an additional 10 seconds to ensure even pellet formation. The supernatant was then removed, and the beads washed with 200 μL of Annealing Buffer (20 mM Tris, pH 7.5 and 5 mM magnesium acetate), vortexed for 5 seconds to resuspend the beads, and pelleted as above. All but approximately 10 μL of the supernatant above the beads were removed, and an additional 200 μL of Annealing Buffer were added. The beads were vortexed again for 5 seconds, allowed to sit for 1 minute, then pelleted as above. All but 10 μL of supernatant were discarded, and 0.48 μL of 2×10⁷molecules per μL template library were added to the beads. The tube was vortexed for 5 seconds to mix the contents, after which the templates were annealed to the beads in a controlled denaturation/annealing program preformed in an MJ thermocycler (5 minutes at 80° C., followed by a decrease by 0.1° C./sec to 70° C., 1 minute at 70° C., decrease by 0.1° C./sec to 60° C., hold at 60° C. for 1 minute, decrease by 0.1° C./sec to 50° C., hold at 50° C. for 1 minute, decrease by 0.1° C./sec to 20° C., hold at 20° C.). Upon completion of the annealing process the beads were stored on ice until needed.

### 4.3 PCR Reaction Mix Preparation and Formulation

To reduce the possibility of contamination, the PCR reaction mix was prepared in a in a UV-treated laminar flow hood located in a PCR clean room. For each 600,000 bead emulsion PCR reaction, 225 μL of reaction mix (1× Platinum HiFi Buffer (Invitrogen), 1 mM dNTPs (Pierce), 2.5 mM MgSO₄ (Invitrogen), 0.1% Acetylated, molecular biology grade BSA (Sigma), 0.01% Tween-80 (Acros Organics), 0.003 U/μL thermostable pyrophosphatase (NEB), 0.625 μM forward (5′-CGTTTCCCCTGTGTGCCTTG-3′ (SEQ ID NO:31)) and 0.039 μM reverse primers (5′-CCATCTGTTGCG TGCGTGTC-3′ (SEQ ID NO:32)) (IDT Technologies, Coralville, Iowa, USA) and 0.15 U/μL Platinum Hi-Fi Taq Polymerase (Invitrogen)) were prepared in a 1.5 mL tube. Twenty-five microliters of the reaction mix were removed and stored in an individual 200 μL PCR tube for use as a negative control. Both the reaction mix and negative controls were stored on ice until needed. Additionally, 240 μL of mock amplification mix (1× Platinum HiFi Buffer (Invitrogen), 2.5 mM MgSO₄ (Invitrogen), 0.1% BSA, 0.01% Tween) for every emulsion were prepared in a 1.5 mL tube, and similarly stored at room temperature until needed.

### 4.4 Emulsification and Amplification

The emulsification process creates a heat-stable water-in-oil emulsion with approximately 10,000 discrete PCR microreactors per microliter which serve as a matrix for single molecule, clonal amplification of the individual molecules of the target library. The reaction mixture and DNA capture beads for a single reaction were emulsified in the following manner: in a UV-treated laminar flow hood, 200 μL of PCR solution were added to the tube containing the 600,000 DNA capture beads. The beads were resuspended through repeated pipette action, after which the PCR-bead mixture was permitted to sit at room temperature for at least 2 minutes, allowing the beads to equilibrate with the PCR solution. Meanwhile, 400 μL of Emulsion Oil (60% (w/w) DC 5225C Formulation Aid (Dow Chemical CO, Midland, Mich.), 30% (w/w) DC 749 Fluid (Dow Chemical CO, Midland, Mich.), and 30% (w/w) Ar20 Silicone Oil (Sigma)) were aliquotted into a flat-topped 2 mL centrifuge tube (Dot Scientific). The 240 μL of mock amplification mix were then added to 400 μL of emulsion oil, the tube capped securely and placed in a 24 well TissueLyser Adaptor (Qiagen) of a TissueLyser MM300 (Retsch GmbH & Co. KG, Haan, Germany). The emulsion was homogenized for 5 minutes at 25 oscillations/sec to generate the extremely small emulsions, or “microfines”, that confer additional stability to the reaction.

During the microfine formation, 160 μL of the PCR amplification mix were added to the mixture of annealed templates and DNA capture beads. The combined beads and PCR reaction mix were briefly vortexed and allowed to equilibrate for 2 minutes. After the microfines had been formed, the amplification mix, templates and DNA capture beads were added to the emulsified material. The TissueLyser speed was reduced to 15 oscillations per second and the reaction mix homogenized for 5 minutes. The lower homogenization speed created water droplets in the oil mix with an average diameter of 100 to 150 μm, sufficiently large to contain DNA capture beads and amplification mix.

The emulsion was aliquotted into 7 to 8 separate PCR tubes each containing roughly 80 μL. The tubes were sealed and placed in a MJ thermocycler along with the 25 μl negative control made previously. The following cycle times were used: 1× (4 minutes at 94° C.)—Hotstart Initiation, 40× (30 seconds at 94° C., 60 seconds at 58° C., 90 seconds at 68° C.)—Amplification, 13× (30 seconds at 94° C., 360 seconds at 58° C.)—Hybridization Extension. After completion of the PCR program, the reactions were removed and the emulsions either broken immediately (as described below) or the reactions stored at 10° C. for up to 16 hours prior to initiating the breaking process.

### 4.5 Breaking the Emulsion and Recovery of Beads

Fifty microliters of isopropyl alcohol (Fisher) were added to each PCR tube containing the emulsion of amplified material, and vortexed for 10 seconds to lower the viscosity of the emulsion. The tubes were centrifuged for several seconds in a microcentrifuge to remove any emulsified material trapped in the tube cap. The emulsion-isopropyl alcohol mix was withdrawn from each tube into a 10 mL BD-Disposable Syringe (Fisher Scientific) fitted with a blunt 16 gauge blunt needle (Brico Medical Supplies). An additional 50 μL of isopropyl alcohol were added to each PCR tube, vortexed, centrifuged as before, and added to the contents of the syringe. The volume inside the syringe was increased to 9 mL with isopropyl alcohol, after which the syringe was inverted and 1 mL of air was drawn into the syringe to facilitate mixing the isopropanol and emulsion. The blunt needle was removed, a 25 mm Swinlock filter holder (Whatman) containing 15 μm pore Nitex Sieving Fabric (Sefar America, Depew, N.Y., USA) attached to the syringe luer, and the blunt needle affixed to the opposite side of the Swinlock unit.

The contents of the syringe were gently but completely expelled through the Swinlock filter unit and needle into a waste container with bleach. Six milliliters of fresh isopropyl alcohol were drawn back into the syringe through the blunt needle and Swinlock filter unit, and the syringe inverted 10 times to mix the isopropyl alcohol, beads and remaining emulsion components. The contents of the syringe were again expelled into a waste container, and the wash process repeated twice with 6 mL of additional isopropyl alcohol in each wash. The wash step was repeated with 6 mL of 80% Ethanol/1× Annealing Buffer (80% Ethanol, 20 mM Tris-HCl, pH 7.6, 5 mM Magnesium Acetate). The beads were then washed with 6 mL of 1× Annealing Buffer with 0.1% Tween (0.1% Tween-20, 20 mM Tris-HCl, pH 7.6, 5 mM Magnesium Acetate), followed by a 6 mL wash with picopure water.

After expelling the final wash into the waste container, 1.5 mL of 1 mM EDTA were drawn into the syringe, and the Swinlock filter unit removed and set aside. The contents of the syringe were serially transferred into a 1.5 mL centrifuge tube. The tube was periodically centrifuged for 20 seconds in a minifuge to pellet the beads and the supernatant removed, after which the remaining contents of the syringe were added to the centrifuge tube. The Swinlock unit was reattached to the filter and 1.5 mL of EDTA drawn into the syringe. The Swinlock filter was removed for the final time, and the beads and EDTA added to the centrifuge tube, pelletting the beads and removing the supernatant as necessary.

### 4.6 Second-Strand Removal

Amplified DNA, immobilized on the capture beads, was rendered single stranded by removal of the secondary strand through incubation in a basic melt solution. One mL of freshly prepared Melting Solution (0.125 M NaOH, 0.2 M NaCl) was added to the beads, the pellet resuspended by vortexing at a medium setting for 2 seconds, and the tube placed in a Thermolyne LabQuake tube roller for 3 minutes. The beads were then pelleted as above, and the supernatant carefully removed and discarded. The residual melt solution was then diluted by the addition of 1 mL Annealing Buffer (20 mM Tris-Acetate, pH 7.6, 5 mM Magnesium Acetate), after which the beads were vortexed at medium speed for 2 seconds, and the beads pelleted, and supernatant removed as before. The Annealing Buffer wash was repeated, except that only 800 μL of the Annealing Buffer were removed after centrifugation. The beads and remaining Annealing Buffer were transferred to a 0.2 mL PCR tube, and either used immediately or stored at 4° C. for up to 48 hours before continuing with the subsequent enrichment process.

### 4.7 Enrichment of Beads

Up to this point the bead mass was comprised of both beads with amplified, immobilized DNA strands, and null beads with no amplified product. The enrichment process was utilized to selectively capture beads with sequenceable amounts of template DNA while rejecting the null beads.

The single stranded beads from the previous step were pelleted by 10 second centrifugation in a benchtop mini centrifuge, after which the tube was rotated 180° and spun for an additional 10 seconds to ensure even pellet formation. As much supernatant as possible was then removed without disturbing the beads. Fifteen microliters of Annealing Buffer were added to the beads, followed by 2 μL of 100 μM biotinylated, 40 base HEG enrichment primer (5′ Biotin—18-atom hexa-ethyleneglycol spacer-CGTTTCCCCTGTGTGCCTTGCCATCTGTTCCCTCCCTGTC-3′ (SEQ ID NO:33), IDT Technologies, complementary to the combined amplification and sequencing sites (each 20 bases in length) on the 3′-end of the bead-immobilized template. The solution was mixed by vortexing at a medium setting for 2 seconds, and the enrichment primers annealed to the immobilized DNA strands using a controlled denaturation/annealing program in an MJ thermocycler (30 seconds at 65° C., decrease by 0.1° C./sec to 58° C., 90 seconds at 58° C., and a 10° C. hold).

While the primers were annealing, a stock solution of SeraMag-30 magnetic streptavidin beads (Seradyn, Indianapolis, Ind., USA) was resuspended by gentle swirling, and 20 μL of SeraMag beads were added to a 1.5 mL microcentrifuge tube containing 1 mL of Enhancing Fluid (2 M NaCl, 10 mM Tris-HCl, 1 mM EDTA, pH 7.5). The SeraMag bead mix was vortexed for 5 seconds, and the tube placed in a Dynal MPC-S magnet, pelletting the paramagnetic beads against the side of the microcentrifuge tube. The supernatant was carefully removed and discarded without disturbing the SeraMag beads, the tube removed from the magnet, and 1004 of enhancing fluid were added. The tube was vortexed for 3 seconds to resuspend the beads, and the tube stored on ice until needed.

Upon completion of the annealing program, 100 μL of Annealing Buffer were added to the PCR tube containing the DNA Capture beads and enrichment primer, the tube vortexed for 5 seconds, and the contents transferred to a fresh 1.5 mL microcentrifuge tube. The PCR tube in which the enrichment primer was annealed to the capture beads was washed once with 200 μL of annealing buffer, and the wash solution added to the 1.5 mL tube. The beads were washed three times with 1 mL of annealing buffer, vortexed for 2 seconds, pelleted as before, and the supernatant carefully removed. After the third wash, the beads were washed twice with 1 mL of ice cold enhancing fluid, vortexed, pelleted, and the supernatant removed as before. The beads were then resuspended in 150 μL ice cold enhancing fluid and the bead solution added to the washed SeraMag beads.

The bead mixture was vortexed for 3 seconds and incubated at room temperature for 3 minutes on a LabQuake tube roller, while the streptavidin-coated SeraMag beads bound to the biotinylated enrichment primers annealed to immobilized templates on the DNA capture beads. The beads were then centrifuged at 2,000 RPM for 3 minutes, after which the beads were gently “flicked” until the beads were resuspended. The resuspended beads were then placed on ice for 5 minutes. Following the incubation on ice, cold Enhancing Fluid was added to the beads to a final volume of 1.5 mL. The tube inserted into a Dynal MPC-S magnet, and the beads were left undisturbed for 120 seconds to allow the beads to pellet against the magnet, after which the supernatant (containing excess SeraMag and null DNA capture beads) was carefully removed and discarded.

The tube was removed from the MPC-S magnet, 1 mL of cold enhancing fluid added to the beads, and the beads resuspended with gentle flicking. It was essential not to vortex the beads, as vortexing may break the link between the SeraMag and DNA capture beads. The beads were returned to the magnet, and the supernatant removed. This wash was repeated three additional times to ensure removal of all null capture beads. To remove the annealed enrichment primers and SeraMag beads from the DNA capture beads, the beads were resuspended in 1 mL of melting solution, vortexed for 5 seconds, and pelleted with the magnet. The supernatant, containing the enriched beads, was transferred to a separate 1.5 mL microcentrifuge tube, the beads pelleted and the supernatant discarded. The enriched beads were then resuspended in 1× Annealing Buffer with 0.1% Tween-20. The beads were pelleted on the MPC again, and the supernatant transferred to a fresh 1.5 mL tube, ensuring maximal removal of remaining SeraMag beads. The beads were centrifuged, after which the supernatant was removed, and the beads washed 3 times with 1 mL of 1× Annealing Buffer. After the third wash, 800 μL of the supernatant were removed, and the remaining beads and solution transferred to a 0.2 mL PCR tube.

The average yield for the enrichment process was 33% of the original beads added to the emulsion, or 198,000 enriched beads per emulsified reaction. As the 60×60 mm PTP format required 900,000 enriched beads, five 600,000 bead emulsions were processed per 60×60 mm PTP sequenced.

### 4.8 Sequencing Primer Annealing

The enriched beads were centrifuged at 2,000 RPM for 3 minutes and the supernatant decanted, after which 15 μL of annealing buffer and 3 μL of sequencing primer (100 mM SAD1F (5′-GCC TCC CTC GCG CCA-3′ (SEQ ID NO:34), IDT Technologies), were added. The tube was then vortexed for 5 seconds, and placed in an MJ thermocycler for the following 4 stage annealing program: 5 minutes at 65° C., decrease by 0.1° C./sec to 50° C., 1 minute at 50° C., decrease by 0.1° C./sec to 40° C., hold at 40° C. for 1 minute, decrease by 0.1° C./sec to 15° C., hold at 15° C.

Upon completion of the annealing program, the beads were removed from thermocycler and pelleted by centrifugation for 10 seconds, rotating the tube 180°, and spun for an additional 10 seconds. The supernatant was discarded, and 200 μL of annealing buffer were added. The beads were resuspended with a 5 second vortex, and the beads pelleted as before. The supernatant was removed, and the beads resuspended in 100 μL annealing buffer, at which point the beads were quantitated with a Multisizer 3 Coulter Counter. Beads were stored at 4° C. and were stable for at least one week.

### 4.9 Incubation of DNA Beads with Bst DNA Polymerase, Large Fragment and SSB Protein

Bead wash buffer (100 ml) was prepared by the addition of apyrase (Biotage) (final activity 8.5 units/liter) to 1× assay buffer containing 0.1% BSA. The fiber optic slide was removed from picopure water and incubated in bead wash buffer. Nine hundred thousand of the previously prepared DNA beads were centrifuged and the supernatant was carefully removed. The beads were then incubated in 1290 μl of bead wash buffer containing 0.4 mg/mL polyvinyl pyrrolidone (MW 360,000), 1 mM DTT, 175 μg of E. coli single strand binding protein (SSB) (United States Biochemicals) and 7000 units of Bst DNA polymerase, Large Fragment (New England Biolabs). The beads were incubated at room temperature on a rotator for 30 minutes.

### 4.10 Preparation of Enzyme Beads and Micro Particle Fillers

UltraGlow Luciferase (Promega) and Bst ATP sulfurylase were prepared in house as biotin carboxyl carrier protein (BCCP) fusions. The 87-aminoacid BCCP region contains a lysine residue to which a biotin is covalently linked during the in vivo expression of the fusion proteins in E. coli. The biotinylated luciferase (1.2 mg) and sulfurylase (0.4 mg) were premixed and bound at 4° C. to 2.0 mL of Dynal M280 paramagnetic beads (10 mg/mL, Dynal SA, Norway) according to manufacturer's instructions. The enzyme bound beads were washed 3 times in 2000 μL of bead wash buffer and resuspended in 2000 μL of bead wash buffer.

Seradyn microparticles (Powerbind SA, 0.8 μm, 10 mg/mL, Seradyn Inc) were prepared as follows: 1050 μL of the stock were washed with 1000 μL of 1× assay buffer containing 0.1% BSA. The microparticles were centrifuged at 9300 g for 10 minutes and the supernatant removed. The wash was repeated 2 more times and the microparticles were resuspended in 1050 μL of 1× assay buffer containing 0.1% BSA. The beads and microparticles are stored on ice until use.

### 4.11 Bead Deposition

The Dynal enzyme beads and Seradyn microparticles were vortexed for one minute and 1000 μL of each were mixed in a fresh micro centrifuge tube, vortexed briefly and stored on ice. The enzyme/Seradyn beads (1920 μl) were mixed with the DNA beads (1300 μl) and the final volume was adjusted to 3460 μL with bead wash buffer. Beads were deposited in ordered layers. The fiber optic slide was removed from the bead wash buffer and Layer 1, a mix of DNA and enzyme/Seradyn beads, was deposited. After centrifuging, Layer 1 supernatant was aspirated off the fiber optic slide and Layer 2, Dynal enzyme beads, was deposited. This section describes in detail how the different layers were centrifuged.

Layer 1.

A gasket that creates two 30×60 mm active areas over the surface of a 60×60 mm fiber optic slide was carefully fitted to the assigned stainless steel dowels on the jig top. The fiber optic slide was placed in the jig with the smooth unetched side of the slide down and the jig top/gasket was fitted onto the etched side of the slide. The jig top was then properly secured with the screws provided, by tightening opposite ends such that they are finger tight. The DNA-enzyme bead mixture was loaded on the fiber optic slide through two inlet ports provided on the jig top. Extreme care was taken to minimize bubbles during loading of the bead mixture. Each deposition was completed with one gentle continuous thrust of the pipette plunger. The entire assembly was centrifuged at 2800 rpm in a Beckman Coulter Allegra 6 centrifuge with GH 3.8-A rotor for 10 minutes. After centrifugation the supernatant was removed with a pipette.

Layer 2.

Dynal enzyme beads (920 μL) were mixed with 2760 μL of bead wash buffer and 3400 μL of enzyme-bead suspension was loaded on the fiber optic slide as described previously. The slide assembly was centrifuged at 2800 rpm for 10 min and the supernatant decanted. The fiber optic slide is removed from the jig and stored in bead wash buffer until it is ready to be loaded on the instrument.

### 4.12 Sequencing on the 454 Instrument

All flow reagents were prepared in 1× assay buffer with 0.4 mg/mL polyvinyl pyrrolidone (MW 360,000), 1 mM DTT and 0.1% Tween 20. Substrate (300 μM D-luciferin (Regis) and 2.5 μM adenosine phophosulfate (Sigma)) was prepared in 1× assay buffer with 0.4 mg/mL polyvinyl pyrrolidone (MW 360,000), 1 mM DTT and 0.1% Tween 20. Apyrase wash is prepared by the addition of apyrase to a final activity of 8.5 units per liter in 1× assay buffer with 0.4 mg/mL polyvinyl pyrrolidone (MW 360,000), 1 mM DTT and 0.1% Tween 20. Deoxynucleotides dCTP, dGTP and dTTP (GE Biosciences) were prepared to a final concentration of 6.5 μM, α-thio deoxyadenosine triphosphate (dATPαS, Biolog) and sodium pyrophosphate (Sigma) were prepared to a final concentration of 50 μM and 0.1 μM, respectively, in the substrate buffer.

The 454 sequencing instrument consists of three major assemblies: a fluidics subsystem, a fiber optic slide cartridge/flow chamber, and an imaging subsystem. Reagents inlet lines, a multi-valve manifold, and a peristaltic pump form part of the fluidics subsystem. The individual reagents are connected to the appropriate reagent inlet lines, which allows for reagent delivery into the flow chamber, one reagent at a time, at a pre-programmed flow rate and duration. The fiber optic slide cartridge/flow chamber has a 250 μm space between the slide's etched side and the flow chamber ceiling. The flow chamber also included means for temperature control of the reagents and fiber optic slide, as well as a light-tight housing. The polished (unetched) side of the slide was placed directly in contact with the imaging system.

The cyclical delivery of sequencing reagents into the fiber optic slide wells and washing of the sequencing reaction byproducts from the wells was achieved by a pre-programmed operation of the fluidics system. The program was written in a form of an Interface Control Language (ICL) script, specifying the reagent name (Wash, dATPαS, dCTP, dGTP, dTTP, and PPi standard), flow rate and duration of each script step. Flow rate was set at 4 mL/min for all reagents and the linear velocity within the flow chamber was approximately ˜1 cm/s. The flow order of the sequencing reagents were organized into kernels where the first kernel consisted of a PPi flow (21 seconds), followed by 14 seconds of substrate flow, 28 seconds of apyrase wash and 21 seconds of substrate flow. The first PPi flow was followed by 21 cycles of dNTP flows (dC-substrate-apyrase wash-substrate dA-substrate-apyrase wash-substrate-dG-substrate-apyrase wash-substrate-dT-substrate-apyrase wash-substrate), where each dNTP flow was composed of 4 individual kernels. Each kernel is 84 seconds long (dNTP-21 seconds, substrate flow-14 seconds, apyrase wash-28 seconds, substrate flow-21 seconds); an image is captured after 21 seconds and after 63 seconds. After 21 cycles of dNTP flow, a PPi kernel is introduced, and then followed by another 21 cycles of dNTP flow. The end of the sequencing run is followed by a third PPi kernel. The total run time was 244 minutes. Reagent volumes required to complete this run are as follows: 500 mL of each wash solution, 100 mL of each nucleotide solution. During the run, all reagents were kept at room temperature. The temperature of the flow chamber and flow chamber inlet tubing is controlled at 30° C. and all reagents entering the flow chamber are pre-heated to 30° C.

### Example 5

### Analysis of Soil Samples

Nucleic acid was extracted from organisms in the soil for analysis using the methods of the invention. Extraction was performed using a DNA extraction kit from Epicentre (Madison, Wis., USA) following manufacturer's directions.

Briefly, 550 ul of Inhibitor Removal Resin was added to each empty Spin Column from Epicentre. The columns were centrifuged for one minute at 2000×g to pack the column. The flow-through was removed and another 550 ul of Inhibitor Removal Resin was added to each column followed by centrifugation for 2 minutes at 2000×g.

100 mg of soil was collected into a 1.5 ml tube and 250 ul of Soil DNA extraction buffer was added with 2 ul of Proteinase K. The solution was vortexed and 50 ul of Soil Lysis buffer was added and vortexed again. The tube was incubated at 65 C for 10 minutes and then centrifuged for 2 minutes at 1000×g. 180 ul of the supernatant was transferred to a new tube and 60 ul of Protein Precipitation Reagent was added with thorough mixing by inverting the tube. The tube was incubated on ice for 8 minutes and centrifuged for 8 minutes at maximum speed. 100-150 ul of the supernatant was transferred directly onto the prepared Spin Column and the column was centrifuged for 2 minutes at 2000×g into the 1.5 ml tube. The column was discarded and the eluate was collected. 6 ul of DNA Precipitation Solution was added to the eluate and the tube was mixed by a brief vortex. Following a 5 minute room temperature incubation, the tube was centrifuged for 5 minutes at maximum speed. Supernatant was removed and the pellet was washed with 500 ul of Pellet Wash Solution. The tube was inverted to mix the solution and then centrifuged for 3 minutes at maximum speed. Supernatant was removed and the wash step was repeated. Supernatant was removed again and the final pellet was resuspended in 300 ul of TE Buffer.

The DNA sample produced may be used for the methods of the invention including, at least, the methods for detecting nucleotide frequency at a locus.

### REFERENCES

BioAnalyzer User Manual (Agilent): hypertext transfer protocol://world wide web.chem.agilent.com/temp/rad31B29/00033620.pdf
BioAnalyzer DNA and RNA LabChip Usage (Agilent): hypertext transfer protocol://world wide web.agilent.com/chem/labonachip
BioAnalyzer RNA 6000 Ladder (Ambion): hypertext transfer protocol://world wide web.ambion.com/techlib/spec/sp_{—}7152.pdf
Biomagnetic Techniques in Molecular Biology, Technical Handbook, 3rd edition (Dynal, 1998): hypertext transfer protocol://world wide web.dynal.no/kunder/dynal/DynalPub36.nsf/cb927fbab127a0ad4125683b004b011c/4908 f5b1a665858a41256adf005779f2/$FILE/Dynabeads M-280 Streptavidin.pdf.
Dinauer et al., 2000 Sequence-based typing of HLA class II DQB1. Tissue Antigens 55:364.
Garcia-Martinez, J., I. Bescos, et al. (2001). “RISSC: a novel database for ribosomal 16S-23S RNA genes spacer regions.” Nucleic Acids Res 29(1): 178-80.
Grahn, N., M. Olofsson, et al. (2003). “Identification of mixed bacterial DNA contamination in broad-range PCR amplification of 16S rDNA V1 and V3 variable regions by pyrosequencing of cloned amplicons.” FEMS Microbiol Lett 219(1): 87-91.
Hamilton, S.C., J. W. Farchaus and M. C. Davis. 2001. DNA polymerases as engines for biotechnology. BioTechniques 31:370.
Jonasson, J., M. Olofsson, et al. (2002). “Classification, identification and subtyping of bacteria based on pyrosequencing and signature matching of 16S rDNA fragments.” Apmis 110(3): 263-72.
MinElute kit (QIAGEN): hypertext transfer protocol://world wide web.qiagen.com/literature/handbooks/minelute/1016839_HBMinElute_Prot_Gel.pdf.
Monstein, H., S, Nikpour-Badr, et al. (2001). “Rapid molecular identification and subtyping of Helicobacter pylori by pyrosequencing of the 16S rDNA variable V1 and V3 regions.” FEMS Microbiol Lett 199(1): 103-7.
Norgaard et al., 1997 Sequencing-based typing of HLA-A locus using mRNA and a single locus-specific PCR followed by cycle-sequencing with AmpliTaq DNA polymerse. Tissue Antigens. 49:455-65.
Pollard, K. S, and M. J. van der Laan (2005). “Clsuter Analysis of Genomic Data with Applications in R.” U.C. Berkeley Division of Biostatistics Working Paper Series #167.
QiaQuick Spin Handbook (QIAGEN, 2001): hypertext transfer protocol://world wide web.qiagen.com/literature/handbooks/qqspin/1016893HBQQSpin_PCR_mc_prot.pdf.
Quick Ligation Kit (NEB): hypertext transfer protocol://world wide web.neb.com/neb/products/mod_enzymes/M2200.html.
Shimizu et al., 2002 Universal fluorescent labeling (UFL) method for automated microsatellite analysis. DNA Res. 9:173-78.
Steffens et al., 1997 Infrared fluorescent detection of PCR amplified gender identifying alleles. J Forensic Sci. 42:452-60.
Team, R. D.C. (2004). R: A language and environment for statistical computing. Vienna, Austria, R Foundation for Statistical Computing.
Tsang et al., 2004 Development of multiplex DNA electronic microarray using a universal adaptor system for detection of single nucleotide polymorphisms. Biotechniques 36:682-88.

We claim:

1. A method for detecting one or more sequence variants in a nucleic acid population comprising the steps of:
(a) amplifying a DNA segment common to said nucleic acid population with a pair of nucleic acid primers that define a locus to produce a first population of amplicons each comprising said DNA segment;
(b) clonally amplifying each member of said first population of amplicons to produce a plurality of populations of second amplicons wherein each population of second amplicons derives from one member of said first population of amplicons;
(c) immobilizing said second amplicons to a plurality of mobile solid support such that each mobile solid support comprises one population of said second amplicons;
(d) determining a nucleic acid sequence for the second amplicons on each solid support to produce a population of nucleic acid sequences;
(e) determining an incidence of each type of nucleotide at each position of said DNA segment to detect the one or more sequence variant in said nucleic acid population.

2. The method of claim 1 wherein said primer is a bipartite primer comprising a 5′ region and a 3′ region, wherein said 3′ region is complementary to a region on said DNA segment and wherein said 5′ region is homologous to a sequencing primer or complement thereof.

3. The method of claim 2 wherein said 5′ region is homologous to a capture oligonucleotide or a complement thereof on said mobile solid support.

4. The method of claim 1 wherein said amplification is performed by polymerase chain reaction.

5. The method of claim 1 wherein said mobile solid support are beads with a diameter selected from the group consisting of between about 1 to about 500 microns, between about 5 to about 100 microns, between about 10 to about 30 microns and between about 15 to about 25 microns.

6. The method of claim 1 wherein said mobile solid support comprise an oligonucleotide which hybridizes and immobilize said first population of amplicons, second amplicons, or both.

7. The method of claim 1 wherein said step of determining a nucleic acid sequence is performed by delivering the plurality of mobile solid supports to an array of at least 10,000 reaction chambers on a planar surface, wherein a plurality of the reaction chambers comprise no more than a single mobile solid support; and determining a nucleic acid sequence of the amplicons on each said mobile solid support.

8. The method of claim 1 wherein said step of determining a nucleic acid sequence is performed by pyrophosphate based sequencing.

9. The method of claim 1 wherein said sequence variant has a frequency selected from the group consisting of less than about 50%, less than about 10%, less than about 5%, less than about 2%, less than about 1%, less than about 0.5%, and less than about 0.2%.

10. The method of claim 1 wherein said sequence variant has a frequency of between 0.2 and 5%.

11. The method of claim 1 wherein said nucleic acid population comprises DNA, RNA, cDNA or a combination thereof.

12. The method of claim 1 wherein the nucleic acid population is derived from a plurality of organisms.

13. The method of claim 1 wherein the nucleic acid population is derived from one organism.

14. The method of claim 13 wherein said nucleic acid population is derived from multiple tissue samples of said organism.

15. The method of claim 13 wherein said nucleic acid population is derived from a single tissue of said organism.

16. The method of claim 1 wherein the nucleic acid population is from a diseased tissue.

17. The method of claim 16 wherein said diseased tissue comprises tumor tissue.

18. The method of claim 1 wherein said nucleic acid population is derived from a bacterial culture, viral culture, or environmental sample.

19. The method of claim 1 wherein the first population of amplicons is 30 to 500 bases in length.

20. The method of claim 1 wherein said first population of amplicons comprises more than 1000 amplicons, more than 5000 amplicons, or more than 10000 amplicons.

21. The method of claim 1 wherein each of said beads binds at least 10,000 members of said plurality of second amplicons.

22. The method of claim 1 wherein the nucleic acid sequence of said DNA segment is undetermined or partially undetermined before said method.

23. A method of identifying a population comprising a plurality of different individual organisms comprising the steps of:
(a) isolating a nucleic acid sample from said population;
(b) determining one or more sequence variant of a nucleic acid segment comprising a locus common to all organisms in said population using the method of claim 1, wherein each organism comprise a different nucleic acid sequence at said locus; and
(c) determining a distribution of organisms in said population based on said population of nucleic acid sequences.

24. The method of claim 23 wherein said population is a population of organisms selected from the group consisting of bacteria, viruses, unicellular organisms, plants and yeasts.

25. A method for determining a composition of a tissue sample comprising the steps of:
(a) isolating a nucleic acid sample from said tissue sample;
(b) detecting a sequence variant of a nucleic acid segment using the method of claim 1, wherein said segment comprises a locus common to all cells in said tissue sample and wherein each cell type comprises a different sequence variant at said locus; and
(c) determining the composition of said tissue sample from said nucleotide frequency.

26. An automated method for genotyping an organism comprising the steps of:
(a) isolating a nucleic acid from said organism;
(b) determining a nucleic acid sequence at one or more loci in said nucleic acid according to the method of claim 1 to produce the population of nucleic acid sequences at that one or more loci;
(c) determining a homozygosity or heterozygosity at said one or more loci from said population of nucleic acid sequences to determine the genotype of said organism.

27. The method of claim 26 further comprising the step of (d) comparing said population of nucleic acid sequence with the sequence of one or more reference genotypes to determine a genotype of said organism.

28. The method of claim 26 wherein said one or more loci comprises SNPs and wherein said genotype is a SNP genotype.


SEQUENCE LISTING (34 sequences)

SEQ ID NO: 1 - DNA, 41 bp, Artificial Sequence
  Synthesized Oligonucleotides
gcctccctcgcgccatcagacctccctctgtgtccttacaa

SEQ ID NO: 2 - DNA, 41 bp, Artificial Sequence
  Synthesized Oligonucleotides
gccttgccagcccgctcagggagggaatcatactagcacca

SEQ ID NO: 3 - DNA, 43 bp, Artificial Sequence
  Synthesized Oligonucleotides
gcctccctcgcgccatcagtctgacgatctctgtcttctaacc

SEQ ID NO: 4 - DNA, 39 bp, Artificial Sequence
  Synthesized Oligonucleotides
gccttgccagcccgctcaggccttgaactacacgtggct

SEQ ID NO: 5 - DNA, 39 bp, Artificial Sequence
  Synthesized Oligonucleotides
gcctccctcgcgccatcagatttctctaccacccctggc

SEQ ID NO: 6 - DNA, 39 bp, Artificial Sequence
  Synthesized Oligonucleotides
gccttgccagcccgctcagagctcatgtctcccgaagaa

SEQ ID NO: 7 - DNA, 39 bp, Artificial Sequence
  Synthesized Oligonucleotides
gcctccctcgcgccatcagaaagccagaagaggaaaggc

SEQ ID NO: 8 - DNA, 39 bp, Artificial Sequence
  Synthesized Oligonucleotides
gccttgccagcccgctcagcttgcagattggtcataagg

SEQ ID NO: 9 - DNA, 39 bp, Artificial Sequence
  Synthesized Oligonucleotides
gcctccctcgcgccatcagacagtgcaaacaccaccaaa

SEQ ID NO: 10 - DNA, 39 bp, Artificial Sequence
  Synthesized Oligonucleotides
gccttgccagcccgctcagccagtattcatggcagggtt

SEQ ID NO: 11 - DNA, 15 bp, Artificial Sequence
  Synthesized Oligonucleotides
gcctccctcgcgcca

SEQ ID NO: 12 - DNA, 15 bp, Artificial Sequence
  Synthesized Oligonucleotides
gccttgccagcccgc

SEQ ID NO: 13 - DNA, 41 bp, Artificial Sequence
  Synthesized Oligonucleotides
gcctccctcgcgccatcaggaagagtttgatcatggctcag

SEQ ID NO: 14 - DNA, 39 bp, Artificial Sequence
  Synthesized Oligonucleotides
gccttgccagcccgctcagttactcacccgtccgccact

SEQ ID NO: 15 - DNA, 39 bp, Artificial Sequence
  Synthesized Oligonucleotides
gcctccctcgcgccatcaggcaacgcgaagaaccttacc

SEQ ID NO: 16 - DNA, 39 bp, Artificial Sequence
  Synthesized Oligonucleotides
gccttgccagcccgctcagacgacagccatgcagcacct

SEQ ID NO: 17 - DNA, 72 bp, Artificial Sequence
  Synthesized Oligonucleotides
aagagttttgatcatggctcagattgaacgctggcggcaggcctaacacatgcaagtcgaacggtaacagga

SEQ ID NO: 18 - DNA, 11 bp, Artificial Sequence
  Synthesized Oligonucleotides
acgaggaacga

SEQ ID NO: 19 - DNA, 10 bp, Artificial Sequence
  Synthesized Oligonucleotides
acaggaacga

SEQ ID NO: 20 - DNA, 72 bp, Artificial Sequence; identical to SEQ ID NO: 17
  Synthesized Oligonucleotides

SEQ ID NO: 21 - DNA, 11 bp, Artificial Sequence
  Synthesized Oligonucleotides
cggtaacagga

SEQ ID NO: 22 - DNA, 11 bp, Artificial Sequence
  Synthesized Oligonucleotides
cggtaacagga

SEQ ID NO: 23 - DNA, 104 bp, Artificial Sequence
  Synthesized Oligonucleotides
caacgcgaagaaccttacctggtcttgacatccacgaagtttactagagatgagaatgtgccgttcgggaaccggtgagacaggtgctgcatggctgtcgtctg

SEQ ID NO: 24 - DNA, 42 bp, Artificial Sequence
  Synthesized Oligonucleotides
ccgttcgggaaccggtgagacaggtgctgcatggctgtcgtc

SEQ ID NO: 25 - DNA, 40 bp, Artificial Sequence
  Synthesized Oligonucleotides
ccttcgggaaccgtgagacaggtgctgcatggctgtcgtc

SEQ ID NO: 26 - DNA, 102 bp, Artificial Sequence
  Synthesized Oligonucleotides
caacgcgaagaaccttacctggtcttgacatccacgaagtttacagagatgagaatgtgccgttcgggaaccgtgagacaggtgctgcatggctgtcgtctg

SEQ ID NO: 27 - DNA, 40 bp, Artificial Sequence
  Synthesized Oligonucleotides
cgttcgggaaccgtgagacaggtgctgcatggctgtcgtc

SEQ ID NO: 28 - DNA, 39 bp, Artificial Sequence
  Synthesized Oligonucleotides
cttcgggaaccgtgagacaggtgctgcatggctgtcgtc

SEQ ID NO: 29 - skipped

SEQ ID NO: 30 - DNA, 20 bp, Artificial Sequence
  Synthesized Oligonucleotides
ccatctgttgcgtgcgtgtc

SEQ ID NO: 31 - DNA, 20 bp, Artificial Sequence
  Synthesized Oligonucleotides
cgtttcccctgtgtgccttg

SEQ ID NO: 32 - DNA, 20 bp, Artificial Sequence
  Synthesized Oligonucleotides
ccatctgttgcgtgcgtgtc

SEQ ID NO: 33 - DNA, 40 bp, Artificial Sequence
  Synthesized Oligonucleotides
cgtttcccctgtgtgccttgccatctgttccctccctgtc

SEQ ID NO: 34 - DNA, 15 bp, Artificial Sequence
  Synthesized Oligonucleotides
gcctccctcgcgcca